(19) United States
(12) Patent Application Publication
(10) Pub. No.: US 2025/0369953 A1
(43) Pub. Date: Dec. 4, 2025

(54) METHODS FOR MEASURING CIRCADIAN RHYTHM FACTORS

(71) Applicant: Evan RAIEWSKI, San Diego, CA (US)

(72) Inventor: Evan RAIEWSKI, San Diego, CA (US)

(21) Appl. No.: 19/221,192

(22) Filed: May 28, 2025

Related U.S. Application Data
(60) Provisional application No. 63/653,590, filed on May 30, 2024.

(51) Int. Cl. G01N 33/493

(52) U.S. Cl. CPC G01N 33/493

(57) ABSTRACT

The technology relates in part to methods for measuring circadian rhythm factors. In some aspects, the technology relates to measuring circadian rhythm factor metabolites. In some aspects, the technology relates to measuring melatonin metabolites. In some aspects, the technology relates to measuring melatonin metabolites and estimating circadian rhythm phase markers according to a distribution function.

### RELATED PATENT APPLICATIONS

This patent application claims the benefit of U.S. provisional patent application No. 63/653,590 filed on May 30, 2024, entitled METHODS FOR MEASURING CIRCADIAN RHYTHM FACTORS, naming Evan RAIEWSKI as inventor, and designated by attorney docket no. RAIEW-1001PROV. The entire content of the foregoing patent application is incorporated herein by reference for all purposes, including all text, tables and drawings.

### FIELD

The technology relates in part to methods for measuring circadian rhythm factors. In some aspects, the technology relates to measuring circadian rhythm factor metabolites. In some aspects, the technology relates to measuring melatonin metabolites. In some aspects, the technology relates to measuring melatonin metabolites and estimating circadian rhythm phase markers according to a distribution function.

### BACKGROUND

Circadian rhythm, or circadian cycle, generally refers to a natural oscillation that repeats periodically (e.g., roughly every 24 hours). Circadian rhythm may refer to a process that originates within an organism (i.e., endogenous) and responds to the environment (is entrained by the environment). Circadian rhythms are regulated by a circadian clock whose primary function is to rhythmically coordinate biological processes so they occur at the correct time to maximize the fitness of an individual. Circadian rhythms have been widely observed in animals, plants, fungi and cyanobacteria.

Melatonin (N-acetyl-5-methoxytryptamine) is a hormone of the pineal gland and is considered a circadian rhythm factor. Melatonin has been associated with several disorders or physiological problems including depression, sleep disturbances, migraine attacks, regulation of the immune system, weight regulation, and regulation of reproduction. In particular, the human circadian rhythm (i.e., the 24-hour biological clock) is highly regulated and dependent on a daily light-dark cycle. Melatonin produced during the night phase of the circadian rhythm can be used to establish suspected problems in the patient's circadian rhythm, in certain applications. Melatonin may be given to humans to treat the phenomenon of “jet lag” following airplane trips associated with a change in time zones. Melatonin also has been given to patients with insomnia, Parkinson disease, and seasonal affective disorders. Melatonin can reduce the time awake before sleep onset, diminish sleep latency and number of awakenings, increase overall sleep efficiency, and improve mood, drive, alertness, and reaction time during the day.

In healthy young adult humans, melatonin generally is secreted as a broad pulse during nighttime sleep in the total amount of approximately 25-30 μg per night, typically producing peak plasma concentrations of approximately 70 μg/ml, occurring at approximately 02:00 am. Melatonin is secreted into the blood stream and may also be secreted into cerebrospinal fluid (CSF). Terminal plasma elimination half-life can range from 20 to 50 minutes, volume of distribution is approximately 40 liters, and the metabolic clearance of melatonin is approximately 1 liter per minute. The primary metabolic pathway transforms melatonin into 6-hydroxymelatonin, which is then conjugated with sulfate to form 6-sulfatoxymelatonin (aMT6s) and excreted in urine as a waste product.

Detection of melatonin in humans can be performed on specific sample types such as saliva or extracted plasma samples using immunological or HLPC detection technologies, for example. An immunological detection of melatonin typically relies on specific antibodies reactive towards melatonin, which are incubated together with melatonin conjugates or melatonin radioactive labels to determine the amount of captured melatonin from samples. Another approach is to measure 6-sulfatoxymelatonin (aMT6s), a urinary metabolite of melatonin. A relationship has been observed between serum or plasma melatonin levels and aMT6s in 24 h urine samples in healthy volunteers. Accordingly, measurement of aMT6s in urine can provide a robust, simple, and reliable assessment of melatonin secretion. Measurement of aMT6s is a noninvasive method to study melatonin given repeated urine fractions can be obtained during a long period without disturbing the subject with repeated blood draws. Provided herein are methods for measuring aMT6s in urine samples and estimating circadian rhythm phase markers according to a distribution function.

### SUMMARY

Provided in certain aspects are methods comprising a) obtaining circadian rhythm factor metabolite measurements from samples from a subject, where the measurements are taken at a plurality of collection times; b) generating a Z-score for each collection time according to the corresponding circadian rhythm factor metabolite measurement; c) generating a normal distribution according to the Z-scores generated in (b) and the plurality of collection times in (a); and d) generating values for one or more phase markers according to the normal distribution in (c).

Also provided in certain aspects are systems comprising one or more microprocessors and memory, which memory comprises instructions executable by the one or more microprocessors and which memory comprises circadian rhythm factor metabolite measurements from samples from a subject, where the measurements are taken at a plurality of collection times, and where the instructions executable by the one or more microprocessors are configured to: a) generate a Z-score for each collection time according to the corresponding circadian rhythm factor metabolite measurement; b) generate a normal distribution according to the Z-scores generated in (a) and the plurality of collection times; and c) generate values for one or more phase markers according to the normal distribution in (b).

Also provided in certain aspects are machines comprising one or more microprocessors and memory, which memory comprises instructions executable by the one or more microprocessors and which memory comprises circadian rhythm factor metabolite measurements from samples from a subject, where the measurements are taken at a plurality of collection times, and where the instructions executable by the one or more microprocessors are configured to a) generate a Z-score for each collection time according to the corresponding circadian rhythm factor metabolite measurement; b) generate a normal distribution according to the Z-scores generated in (a) and the plurality of collection times; and c) generate values for one or more phase markers according to the normal distribution in (b).

Also provided in certain aspects is a non-transitory computer-readable storage medium with an executable program stored thereon, where the program instructs a microprocessor to perform the following a) access circadian rhythm factor metabolite measurements from samples from a subject, where the measurements are taken at a plurality of collection times; b) generate a Z-score for each collection time according to the corresponding circadian rhythm factor metabolite measurement; c) generate a normal distribution according to the Z-scores generated in (b) and the plurality of collection times in (a); and d) generate values for one or more phase markers according to the normal distribution in (c).

Certain implementations are described further in the following description, examples and claims, and in the drawings.

### BRIEF DESCRIPTION OF THE DRAWINGS

The drawings illustrate certain implementations of the technology and are not limiting. For clarity and ease of illustration, the drawings are not made to scale and, in some instances, various aspects may be shown exaggerated or enlarged to facilitate an understanding of particular implementations.

FIG. 1 shows example equations for generating parameters of the parent normal distribution. The estimation of sigma (Equation A) involves the time and Z-scores associated with the beginning (i-1) and end (i) of the collection interval containing the midpoint (e.g., cumulative proportion of 0.5, when Z=0). Sigma computation is shown in Computation A. Estimation of mu is shown in Equation B and mu computation is shown in Computation B. Equation C depicts the standard function producing the normal distribution; Equation D displays a modified variant producing the parent normal distribution, using estimated mu and sigma and multiplying the entire function by the grand nightly aMT6s total (in this example, 7780.963). This last operation sets the area under the curve of the parent normal distribution equal to the grand nightly total of excreted aMT6s.

FIG. 2 shows parent normal distribution is the central feature of the Z distribution fit (Raiewski Fit). Parameters μ (27.845 h), σ (2.036 h) and total nightly aMT6s ng count (7780.97 ng) define properties of the underlying parent normal distribution. Known proportions of the normal distribution curve allow for determining the peak, or maximum, value (1525 ng) occurring at time p. These coordinates (27.845, 1525) are commonly referred to as peak coordinates. Time (h) of the peak is analogous to the acrophase in trigonometric (i.e., sin, cosine) terminology.

FIG. 3 shows fitted aMT6s ng/h values estimate actual aMT6s ng/h data. Proportions of total nightly aMT6s ng can be determined within each collection interval defined by actual collection times. This number represents the total aMT6s ng collected at each interval. When divided by the time interval (h) results in the fitted aMT6s ng/h that ultimately predict aMT6s ng/h values of real data. Unlike the parent normal distribution used to compute these fitted values, fitted data in ng/h match units of measure going into the Z distribution fit (Raiewski Fit) algorithm (parent normal distribution is more accurately described as a histogram, reflecting counts of ng excreted at a selected time). Goodness of fit is assessed in two ways, first taking the Pearson's correlation coefficient, r, among real and fitted datapoints at shared timepoints, and second, taking the residual standard deviation (RSD) among real and fitted datapoints, quantifying the typical residual, or difference, among real and fitted datapoints.

FIG. 4 shows an existing cosine fit approach over 3 days, 1.5 h collection interval schedule. Dataset is partitioned into three separate 24 h intervals at trough (minimum) values (˜hour 40 and 64) for analysis using Z distribution fit (Raiewski Fit). A indicates maximum values predicted by cosine fit function=899.25 ng/h, reliably underestimating nightly maximum values. Each acrophase is 24 h apart, eliminating sensitivity to intra-day variation. B indicates 100% Mesor value=342.75 ng/h. 100% Mesor values are the standard marker for determination of onset and offset where actual aMT6s ng/h values cross above and below this threshold. One issue with this method can be seen near hour 30 where data briefly dips below and back above threshold before truly returning to basal levels for the day, producing errant calculation and an invitation to misinterpreting results. C indicates minimum values produced by cosine fit function=−213.75 ng/h. Cosine fit determined minimum values are biologically unrealistic: real aMT6s waveform maintains prolonged basal (near zero) levels and cannot go below zero.

FIG. 5 shows 3 consecutive days of fitted ng/h data from actual aMT6s ng/h. Parent normal distribution generating fitted ng/h values are not shown, but the determined parameters are displayed. As previously described, data are partitioned using trough times from non-fixed 24 h cosine fit (˜hour 40 and hour 64, above). Once partitioned, the Z distribution fit (Raiewski Fit) is run on each segment separately. Data are reintegrated here for visualization purposes.

FIG. 6 shows determining phase markers of onset and offset with the parent normal distribution. The cosine fit model (see Night 1, FIG. 4) commonly estimates circadian onset and offset by determining when actual aMT6s ng/h first cross above and last cross below a “threshold” value. This value is typically the mesor (vertical midpoint of the fitted cosine wave). The vertical midpoint of the parent normal distribution (above, 762.4) is 50% the peak value (1524.78) and serves as the closest equivalent method of estimating onset and offset in comparison to the cosine fit method. In FIG. 5, actual ng/h data cross this threshold at 26.122 h and 30.292 h. This threshold crosses the parent normal distribution at 25.448 h and 30.242 h, where Z=+/−1.177. Onset and offset taken from fitted ng/h data crossing this threshold are 26.168 h and 30.905 h, respectively.

FIG. 7 shows aMT6s profiles of real data are highly sensitive to collection interval. Using parameters from the example parent normal distribution (μ=26.25 h, σ=3.0 h, nightly total aMT6s ng count=12,000 ng), an array of resulting aMT6s ng/h profiles intended to simulate results of real data collection are shown above, sampled at 1, 1.5, 2, 4, 6, and 8 hr intervals. As collection intervals increase, real data lose resolution and become less representative of the underlying rhythm researchers wish to uncover. As the collection interval increases, fewer datapoints are obtained, and the max value occurs proportionately later and with attenuated height.

FIG. 8 shows cosine fit results are highly influenced by collection interval. An array of cosine fit from hypothetical data collected from the example parent normal distribution discussed above (μ=26.25 h, σ=3.0 h, nightly total aMT6s count=12,000 ng) at 1, 1.5, 2, 4, 6, and 8 h intervals.

FIG. 9 shows valuable parameters can be extrapolated from the parent normal distribution. The example parent normal distribution is plotted (above) by choosing parameters of μ (26.25 h i.e., 02:15), σ (3.0 h), and a nightly total aMT6s count (12,000 ng). Utilizing the function described FIG. 2, Equation D, with these parameters, peak value (height at μ=26.25 h) equals 1596 ng and produces a 50% peak threshold of 798 ng. The parent normal distribution crosses this threshold at 22.72 h (onset) and 29.78 h (offset), and these times occur at times corresponding to Z+/−1.177. Taking further advantage of Z-scores by using associated proportions under a normal distribution (i.e., Z table) reveals the elevated duration (time between onset and offset, 7.09 h) accounts for a proportion of 0.761 of the total nightly aMT6s excretion (9128.70 ng). Importantly, Z-scores of +/−1.177 and their proportions can be applied in general toward any parent normal distribution. In other words, a 50% peak threshold will always occur at Z +/−1.177 and capture the middle 76.1% of the nightly total aMT6s ng excretion.

FIG. 10 shows taking advantage of the Z distribution and associated proportions to improve phase marker estimates. Rather than selecting an arbitrary percent of peak height to locate crossing points estimating onset and offset, a “middle proportion” threshold may yield superior estimates. Sigma (σ) is the inherent parameter defining the width of a normal distribution. Thresholds determined by 1, 2, and 3 σ from u are shown above, in addition to corresponding middle proportions and percent peak heights at those locations.

FIG. 11 shows in panel A, 3 consecutive days of fitted ng/h data resulting from parent normal distribution (parameters shown). FIG. 11 shows in panel B, fitted ng/h from recomputed parent normal distribution after merging errant low points with proceeding high points. Results are robust when errant data do not contain μ. When errant data contain μ (Day 3) the resulting fit of merged data returns improved, lower intra-day variation, and in all 3 days, facilitate more reliable onset-offset estimates.

FIG. 12 shows a Great Z Table.

### DETAILED DESCRIPTION

Provided herein are methods for estimating circadian rhythm phase marker values according to circadian rhythm factor measurements. Provided herein are methods for estimating circadian rhythm phase marker values according to circadian rhythm factor metabolite measurements. In some embodiments, a method comprises generating Z-scores for circadian rhythm factor metabolite measurements. In some embodiments, a method comprises generating a distribution according to Z-scores for circadian rhythm factor metabolite measurements. In some embodiments, a method comprises estimating circadian rhythm phase marker values according to a distribution of Z-scores.

### Circadian Rhythm Factor Measurements

In some embodiments, a method herein comprises obtaining circadian rhythm factor measurements. In some embodiments, a method herein comprises measuring one or more circadian rhythm factors. Circadian rhythm factors generally refer to molecules such as hormones, genes, proteins, and the like that are regulated by a circadian rhythm. Any suitable method for measuring a circadian rhythm factor may be used. In some embodiments, a circadian rhythm factor is melatonin.

In some embodiments, a method herein comprises obtaining circadian rhythm factor metabolite measurements. In some embodiments, a method herein comprises measuring one or more circadian rhythm factor metabolites. Circadian rhythm factor metabolite generally refers to a circadian rhythm factor that has been processed in some way. For example, a circadian rhythm factor metabolite may be an intermediate or end product of a metabolized circadian rhythm factor. Any suitable method for measuring a circadian rhythm factor metabolite may be used. In some embodiments, a circadian rhythm factor metabolite is a metabolite of melatonin. In some embodiments, a circadian rhythm factor metabolite 6-hydroxymelatonin. In some embodiments, a circadian rhythm factor is 6-sulfatoxymelatonin (aMT6s). In some embodiments, an ELISA kit may be used to measure aMT6s. For example, an ELISA kit manufactured by Buhlman Labs and distributed by ALPCO (see World Wide Web Uniform Resource Locator alpco.com/6-sulfatoxymelatonin-elisa-4385.html) may be used, as described in Kripke et al. (2007) Journal of Circadian Rhythms, 5:4; and Youngstedt et al. (2019) Journal of Physiology, 597.8 pp 2253-2268, each of which is incorporated by reference in its entirety. In certain instances, an ELISA kit sold by Novolytics (see World Wide Web Uniform Resource Locator novolytix.ch/en/6_sulfatoxymelatonin) may be used.

In some embodiments, measurements (i.e., circadian rhythm factor measurements or circadian rhythm factor metabolite measurements) are taken at a plurality of collection times. A collection time generally refers to the time at which a sample is collected (e.g., the time at which urine is excreted and collected). Collection times generally include a first collection time, a last collection time, and at least two collection times between the first and last collection times. In some embodiments, a measurement is zero or close to zero at a first collection time. In some embodiments, a measurement is zero or close to zero at a last collection time. In some embodiments, a plurality of collection times comprises four or more collection times. In some embodiments, a plurality of collection times comprises five or more collection times. In some embodiments, a plurality of collection times comprises six or more collection times. In some embodiments, a plurality of collection times comprises seven or more collection times. In some embodiments, a plurality of collection times comprises eight or more collection times. In some embodiments, a plurality of collection times comprises nine or more collection times. In some embodiments, a plurality of collection times comprises ten or more collection times. In some embodiments, a plurality of collection times consists of four collection times. In some embodiments, a plurality of collection times consists of five collection times. In some embodiments, a plurality of collection times consists of six collection times. In some embodiments, a plurality of collection times consists of seven collection times. In some embodiments, a plurality of collection times consists of eight collection times. In some embodiments, a plurality of collection times consists of nine collection times. In some embodiments, a plurality of collection times consists of 10 collection times.

In some embodiments, collection times are at equal intervals. For example, collection times may be every half hour, every hour, every two hours, every three hours, etc. In some embodiments, collection times are at nonequal intervals. For example, collection times may be at 8:00 pm, 10:00 pm, 1:00 am, and 6:00 am. Thus, the nonequal intervals in this example are 2 hours, 3 hours, and 4 hours.

Measurements herein may be taken at a plurality of time points during a collection period. A collection period generally refers to the time between a first measurement and a last measurement. In some embodiments, a collection period is about 24 hours. In some embodiments, a collection period is less than 24 hours. For example, a collection period may be 23 hours or less, 22 hours or less, 21 hours or less, 20 hours or less, 19 hours or less, 18 hours or less, 17 hours or less, 16 hours or less, 15 hours or less, 14 hours or less, 13 hours or less, 12 hours or less, 11 hours or less, 10 hours or less, 9 hours or less, 8 hours or less, 7 hours or less, or 6 hours or less.

### Z-scores

In some embodiments, a method herein comprises generating a standard score (e.g., Z-score, Z-value, normal score, standardized variable). A standard score generally indicates how many standard deviations a datum is above or below a population/sample mean. A standard score may be derived by subtracting a population/sample mean from an individual raw score and then dividing the difference by a population/sample standard deviation. In some embodiments, a method herein comprises generating a Z-score. In some embodiments, a Z-score is generated according to a measurement (i.e., circadian rhythm factor measurement or circadian rhythm factor metabolite measurement) taken a given collection time. In some embodiments, a Z-score is generated for each collection time (e.g., during a collection period) according to a corresponding measurement.

In some embodiments, a method herein comprises generating a cumulative measurement (i.e., circadian rhythm factor measurement or circadian rhythm factor metabolite measurement). In some embodiments, a method herein comprises generating a cumulative measurement for each collection time (e.g., during a collection period). A cumulative measurement adds the measurement value for a collection time to the measurement taken at the previous collection time (e.g., see column D of Table 1). In some embodiments, a method herein comprises generating a total measurement (i.e., circadian rhythm factor measurement or circadian rhythm factor metabolite measurement). In some embodiments, a method herein comprises generating a total measurement for a collection period. A total measurement sums the measurements from each time point and is equal to the final cumulative measurement (e.g., see last row of column D, Table 1). In some embodiments, a method herein comprises generating a cumulative proportion. In some embodiments, a method herein comprises generating a cumulative proportion for each collection time. A cumulative proportion may be generated by dividing each cumulative measurement by the total measurement (e.g., see column E of Table 1).

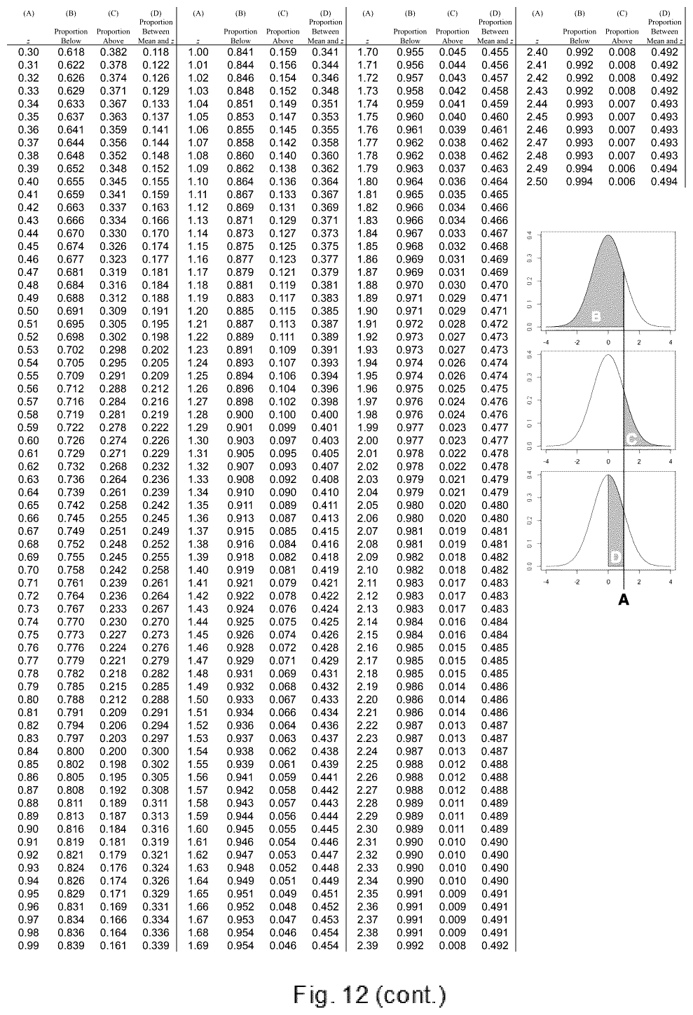

In some embodiments, a Z-score is generated according to a cumulative proportion (e.g., see column F of Table 1). In some embodiments, a Z-score is generated according to a cumulative proportion for each collection time. Accordingly, in some embodiments, a method herein comprises generating a Z-score according to a cumulative proportion for each collection time. Generally, the relationship between cumulative proportions and Z scores (and their fixed location along a normal distribution) are tabulated in a Z table (see Great Z Table in FIG. 12). A user can go to the Z table with a Z score (e.g., given Z=−0.24, find row where Z=−0.24 in Column A), and look up the cumulative proportion associated with that Z score (same row, Column B, cumulative proportion=0.405). Specifically for this workflow, the reverse process may be performed, taking a known cumulative proportion (e.g., cumulative proportion =0.982, find this value in Column B) and determining its Z score counterpart (same row, column A, Z=2.10).

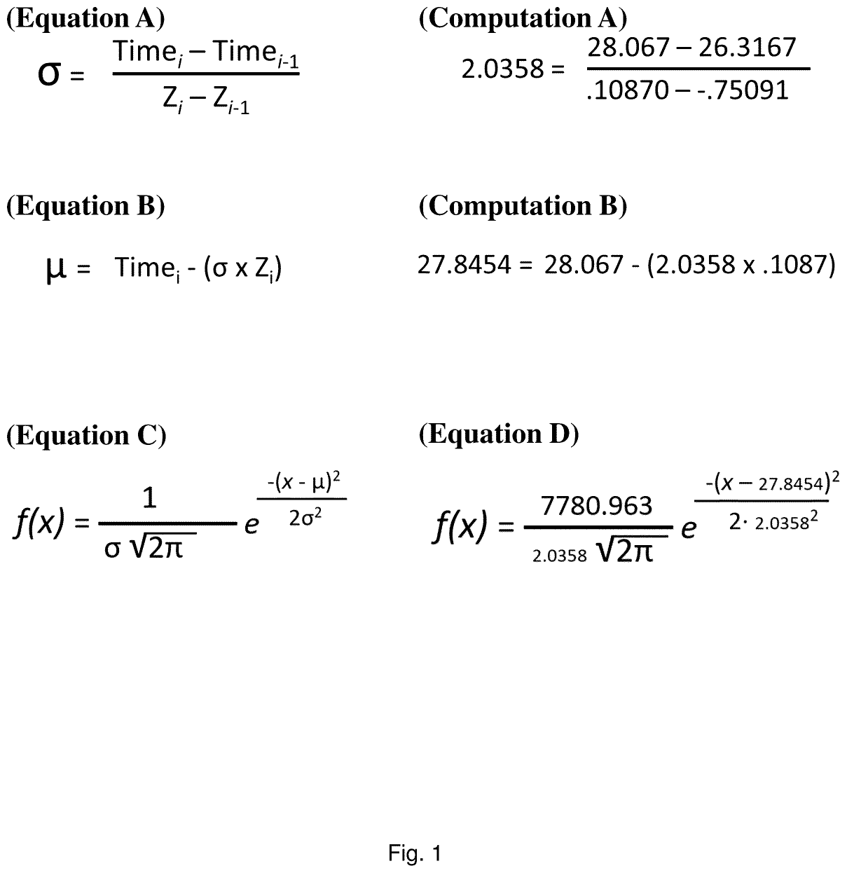

Cumulative proportions may be calculated using integral calculus on the function of a normal distribution (FIG. 1, Equation C), between −∞ and the Z score in question. Illustrated in integral calculus form:

$$f ( x ) = {{\int }^{Z}}_{- ∞} \frac{1}{σ \sqrt{2 π}} {e}^{\frac{- {( x \cdot μ )}^{2}}{2 {σ}^{2}}} dx \qquad Equation E$$

where, given parameters of a standard normal distribution, ρ=0 and σ=1. With regard to the specific example data in Table 1, substituting a value from Column F (Z Score) for the upper range of the interval “Z”, results in the cumulative proportion displayed in Column E. Specifically, selecting any Z score in Column F into the following equation:

$$f ( x ) = {\int }_{- ∞}^{Z} \frac{1}{( \sqrt{2 \cdot π \cdot {1}^{2}} )} {e}^{\frac{- {( x - 0 )}^{2}}{2 \cdot {1}^{2}}} dx \qquad Equation F$$

will return the corresponding cumulative proportion in Column E. Conversely, differential calculus of the same equation would take the cumulative proportion as input and return the corresponding Z score as output. This function may be written as a shortcut in a suitable software program (e.g., desmos, excel, numbers, python, and the like).

### Distribution and Phase Markers

In some embodiments, a method herein comprises generating a distribution (e.g., a distribution curve; a bell curve; a bell-shaped curve; a normal distribution; a normal distribution curve; Gaussian distribution). In some embodiments, a method herein comprises generating a normal distribution. A normal distribution generally refers to a type of continuous probability distribution for a real-valued random variable. A normal distribution may be produced by a normal density function. For example, a normal density function is provided in FIG. 1, Equation C, where the parameter mu (μ) generally refers the mean or expectation of the distribution (and also its median and mode), the parameter sigma (σ) refers to its standard deviation, and σ² refers to the variance of the distribution.

In some embodiments, a distribution is a modified normal distribution. A modified normal distribution may be referred to herein a parent normal distribution. A modified normal distribution may be produced by a modified normal density function (e.g., a function that is a modified form of the function provided by Equation C in FIG. 1). In some embodiments, a modified normal density function includes one or more measured parameters. In some embodiments, the one or more measured parameters comprise total measurement (e.g., total circadian rhythm factor measurement in a collection period or total circadian rhythm factor metabolite measurement in a collection period). A modified normal distribution herein may refer to a normal distribution where the parameters sigma (σ) and mu (μ) in the distribution function are uniquely calculated as described herein, and where a total measurement (e.g., total circadian rhythm factor measurement in a collection period or total circadian rhythm factor metabolite measurement in a collection period) is included in the distribution function. In particular, a modified normal distribution herein refers to a normal distribution where the parameters sigma (σ) and mu (μ) in the distribution function are calculated according to FIG. 1, Equation A, and FIG. 1, Equation B, respectively, and the value 1 in the numerator of the normal distribution function (FIG. 1, Equation C) is replaced by a total measurement (e.g., total circadian rhythm factor measurement in a collection period or total circadian rhythm factor metabolite measurement in a collection period), as in FIG. 1, Equation D.

In some embodiments, a method herein comprises generating a distribution (e.g., a normal distribution; a modified normal distribution) according to Z-scores generated as described herein. In some embodiments, a method herein comprises generating a distribution (e.g., a normal distribution; a modified normal distribution) according to Z-scores generated as described herein and a plurality of collection times. For example, a modified distribution function may comprise parameters determined according to Z-scores generated as described herein and a plurality of collection times. In some embodiments, a modified distribution function comprises parameters sigma (σ) and mu (μ) determined according to Z-scores generated as described herein and a plurality of collection times. In some embodiments, parameters sigma (σ) and mu (μ) are calculated according to FIG. 1, Equation A, and FIG. 1, Equation B, respectively.

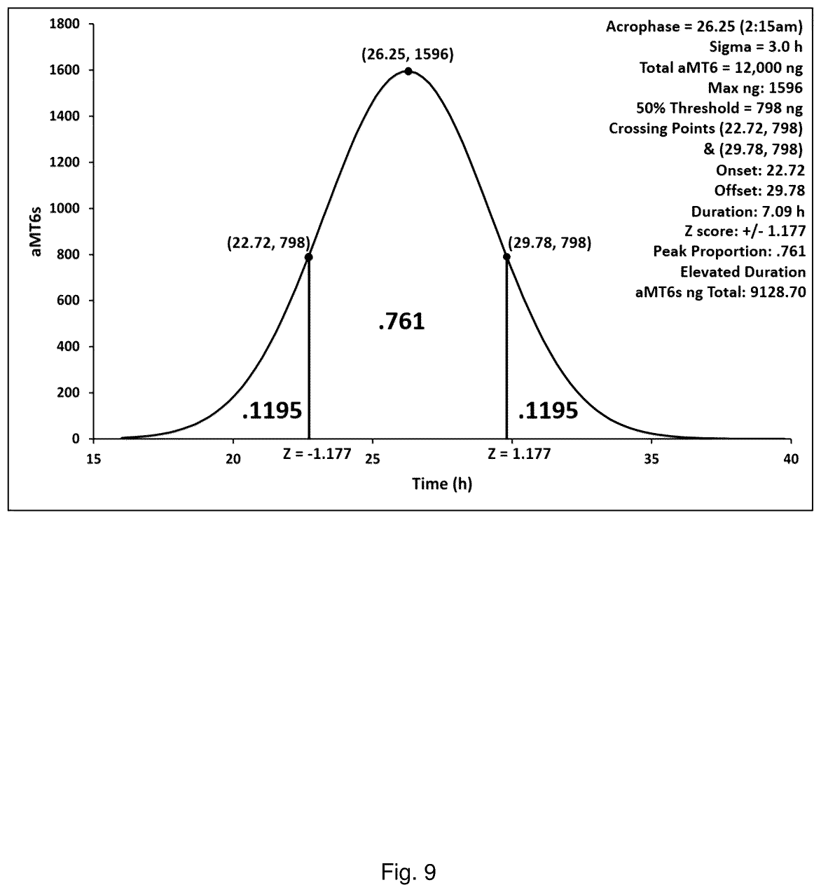

In some embodiments, a method herein comprises generating a value for a phase marker. A phase marker generally refers to a measurable feature of a circadian rhythm factor or circadian rhythm factor metabolite. For example, phase markers may include one or more of onset, offset, duration, peak value, and peak time. Onset generally refers to a rise in quantity of a circadian rhythm factor or circadian rhythm factor metabolite. Offset generally refers to a decline in quantity of a circadian rhythm factor or circadian rhythm factor metabolite. Duration generally refers to an elevated period length for a circadian rhythm factor or circadian rhythm factor metabolite. Peak value generally refers a maximum value of a circadian rhythm factor or circadian rhythm factor metabolite. Peak time generally refers to when a peak value occurred. In some embodiments, a method herein comprises generating values for one or more phase markers. In some embodiments, a method herein comprises generating values for two or more phase markers. In some embodiments, a method herein comprises generating values for three or more phase markers. In some embodiments, a method herein comprises generating values for four or more phase markers. In some embodiments, a method herein comprises generating values for five or more phase markers. A value for a phase marker may be generated according to a normal distribution described herein. In some embodiments, a value for a phase marker is generated according to a modified normal distribution described herein. For example, phase marker values may be estimated according to a modified normal distribution as shown in FIG. 9.

### Samples

Provided herein are methods for analyzing a circadian rhythm factor or circadian rhythm factor metabolite in a sample from a subject. A subject can be any living organism, including but not limited to a human, a non-human animal, a plant, a bacterium, a fungus, a protist or a pathogen. Any human or non-human animal can be selected, and may include, for example, mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. A subject may be a male or female. In some embodiments, a subject is a female. In some embodiments, a subject is a human female. In some embodiments, a subject is a male. In some embodiments, a subject is a human male. A subject may be nonbinary or intersex. A subject may be any age (e.g., an embryo, a fetus, an infant, a child, an adult).

A sample may be any specimen that is isolated or obtained from a subject or part thereof (e.g., a human subject). Non-limiting examples of specimens include fluid or tissue from a subject, including, without limitation, urine, blood or a blood product (e.g., serum, plasma, or the like), umbilical cord blood, chorionic villi, amniotic fluid, cerebrospinal fluid, spinal fluid, lavage fluid (e.g., bronchoalveolar, gastric, peritoneal, ductal, ear, arthroscopic), biopsy sample (e.g., from pre-implantation embryo; cancer biopsy), celocentesis sample, cells (blood cells, placental cells, embryo or fetal cells, fetal nucleated cells or fetal cellular remnants, normal cells, abnormal cells (e.g., cancer cells)) or parts thereof (e.g., mitochondrial, nucleus, extracts, or the like), washings of female reproductive tract, feces, sputum, saliva, nasal mucous, prostate fluid, lavage, semen, lymphatic fluid, bile, tears, sweat, breast milk, breast fluid, the like or combinations thereof. In some embodiments, a biological sample is a cervical swab from a subject.

### Outcomes and Reports

Methods described herein can provide an outcome indicative of one or more characteristics of a sample or subject. For example, methods described herein may provide an outcome indicative of one or more circadian rhythm characteristics for a subject. In some embodiments, an outcome includes a conclusion that predicts and/or determines one or more circadian rhythm characteristics for a subject. In some embodiments, an outcome includes an estimation of one or more phase marker values (e.g., onset, offset, duration, peak value, peak time) as described herein.

Any suitable expression of an outcome can be provided. An outcome sometimes is based on and/or includes one or more numerical values generated according to a method described herein in the context of one or more considerations of probability. Non-limiting examples of values that can be utilized include a sensitivity, specificity, standard deviation, median absolute deviation (MAD), measure of certainty, measure of confidence, measure of certainty or confidence that a value obtained for a sample or subject is inside or outside a particular range of values, measure of uncertainty, measure of uncertainty that a value obtained for a sample or subject is inside or outside a particular range of values, coefficient of variation (CV), confidence level, confidence interval (e.g., about 95% confidence interval), standard score (e.g., Z-score), chi value, phi value, result of a t-test, p-value, area ratio, median level, the like or combination thereof. In some embodiments, an outcome comprises a plot (e.g., a distribution plot). A consideration of probability can facilitate determining one or more characteristics of a sample or subject and/or whether a subject is at risk of having, or has, a disease or disorder (e.g., a disease or disorder associated with circadian rhythms).

In some embodiments, a report may be generated to provide an outcome. In some embodiments a method herein comprises generating a report for one or more phase marker values (e.g., onset, offset, duration, peak value, peak time) as described herein. An outcome for a test subject may be ordered by, and may be provided to, a health care professional or other qualified individual (e.g., physician or assistant) who transmits an outcome to a subject from whom the test sample is obtained. In certain embodiments, an outcome is provided using a suitable visual medium (e.g., a peripheral or component of a machine, e.g., a printer or display). An outcome may be provided to a healthcare professional or qualified individual in the form of a report. A report typically comprises a display of an outcome, may include an associated confidence parameter, and may include a measure of performance for a test used to generate the outcome. A report may include a recommendation for a follow-up procedure (e.g., a procedure that confirms the outcome).

A report can be displayed in a suitable format that facilitates evaluation of a subject's circadian rhythms by a health professional or other qualified individual. Non-limiting examples of formats suitable for use for generating a report include digital data, a graph, a 2D graph, a 3D graph, and 4D graph, a picture (e.g., a jpg, bitmap (e.g., bmp), pdf, tiff, gif, raw, png, the like or suitable format), a pictograph, a chart, a table, a bar graph, a pie graph, a diagram, a flow chart, a scatter plot, a map, a histogram, a density chart, a function graph, a circuit diagram, a block diagram, a bubble map, a constellation diagram, a contour diagram, a cartogram, spider chart, Venn diagram, nomogram, and the like, or combination of the foregoing.

A report may be generated by a computer and/or by human data entry, and can be transmitted and communicated using a suitable electronic medium (e.g., via the internet, via computer, via facsimile, from one network location to another location at the same or different physical sites), or by another method of sending or receiving data (e.g., mail service, courier service and the like). Non-limiting examples of communication media for transmitting a report include auditory file, computer readable file (e.g., pdf file), paper file, laboratory file, medical record file, or any other medium described in the previous paragraph. A laboratory file or medical record file may be in tangible form or electronic form (e.g., computer readable form), in certain embodiments. After a report is generated and transmitted, a report can be received by obtaining, via a suitable communication medium, a written and/or graphical representation comprising an outcome, which upon review allows a healthcare professional or other qualified individual to make a determination as to one or more characteristics of a sample or subject.

An outcome may be provided by and obtained from a laboratory (e.g., obtained from a laboratory file). A laboratory file can be generated by a laboratory that carries out one or more tests for determining one or more characteristics of a sample or subject. Laboratory personnel (e.g., a laboratory manager) can analyze information associated with test samples (e.g., test profiles, reference profiles, test values, reference values, level of deviation, patient information) underlying an outcome. For calls pertaining to presence or absence of an abnormality and/or medical condition that are close or questionable, laboratory personnel can re-run the same procedure using the same (e.g., aliquot of the same sample) or different sample from a subject. A laboratory may be in the same location or different location (e.g., in another country) as personnel assessing the presence or absence of an abnormality and/or medical condition from the laboratory file. For example, a laboratory file can be generated in one location and transmitted to another location in which the information for a sample or subject therein is assessed by a healthcare professional or other qualified individual, and optionally, transmitted to the subject from which the sample was obtained. A laboratory generating a laboratory test report sometimes is a certified laboratory, and sometimes is a laboratory certified under the Clinical Laboratory Improvement Amendments (CLIA).

An outcome sometimes is a component of a diagnosis for a subject, and sometimes an outcome is utilized and/or assessed as part of providing a diagnosis for a subject. For example, a healthcare professional or other qualified individual may analyze an outcome and provide a diagnosis based on, or based in part on, the outcome.

An outcome sometimes is not a component of a diagnosis for a subject and is not utilized and/or assessed as part of providing a diagnosis for a subject. For example, a researcher studying circadian rhythms may use an outcome for research purposes only.

### Machines, Software and Interfaces

Certain processes and methods described herein often are too complex for performing in the mind and cannot be performed without a computer, microprocessor, software, module or other machine. Methods described herein may be computer-implemented methods, and one or more portions of a method sometimes are performed by one or more processors (e.g., microprocessors), computers, systems, apparatuses, or machines (e.g., microprocessor-controlled machine).

Computers, systems, apparatuses, machines and computer program products suitable for use often include, or are utilized in conjunction with, computer readable storage media. Non-limiting examples of computer readable storage media include memory, hard disk, CD-ROM, flash memory device and the like. Computer readable storage media generally are computer hardware, and often are non-transitory computer-readable storage media. Computer readable storage media are not computer readable transmission media, the latter of which are transmission signals per se.

Provided herein are computer readable storage media with an executable program stored thereon, where the program instructs a microprocessor to perform a method described herein. Provided also are computer readable storage media with an executable program module stored thereon, where the program module instructs a microprocessor to perform part of a method described herein. Also provided herein are systems, machines, apparatuses and computer program products that include computer readable storage media with an executable program stored thereon, where the program instructs a microprocessor to perform a method described herein. Provided also are systems, machines and apparatuses that include computer readable storage media with an executable program module stored thereon, where the program module instructs a microprocessor to perform part of a method described herein.

Also provided are computer program products. A computer program product often includes a computer usable medium that includes a computer readable program code embodied therein, the computer readable program code adapted for being executed to implement a method or part of a method described herein. Computer usable media and readable program code are not transmission media (i.e., transmission signals per se). Computer readable program code often is adapted for being executed by a processor, computer, system, apparatus, or machine.

In some embodiments, methods described herein are performed by automated methods. In some embodiments, one or more steps of a method described herein are carried out by a microprocessor and/or computer, and/or carried out in conjunction with memory. In some embodiments, an automated method is embodied in software, modules, microprocessors, peripherals and/or a machine comprising the like, that perform methods described herein. As used herein, software refers to computer readable program instructions that, when executed by a microprocessor, perform computer operations, as described herein.

Machines, software and interfaces may be used to conduct methods described herein. Using machines, software and interfaces, a user may enter, request, query or determine options for using particular information, programs or processes, which can involve implementing statistical analysis algorithms, statistical significance algorithms, statistical algorithms, iterative steps, validation algorithms, and graphical representations, for example. In some embodiments, a data set may be entered by a user as input information, a user may download one or more data sets by suitable hardware media (e.g., flash drive), and/or a user may send a data set from one system to another for subsequent processing and/or providing an outcome.

A system typically comprises one or more machines. Each machine comprises one or more of memory, one or more microprocessors, and instructions. Where a system includes two or more machines, some or all of the machines may be located at the same location, some or all of the machines may be located at different locations, all of the machines may be located at one location and/or all of the machines may be located at different locations. Where a system includes two or more machines, some or all of the machines may be located at the same location as a user, some or all of the machines may be located at a location different than a user, all of the machines may be located at the same location as the user, and/or all of the machine may be located at one or more locations different than the user.

A user may, for example, place a query to software which then may acquire a data set via internet access, and in certain embodiments, a programmable microprocessor may be prompted to acquire a suitable data set based on given parameters. A programmable microprocessor also may prompt a user to select one or more data set options selected by the microprocessor based on given parameters. A programmable microprocessor may prompt a user to select one or more data set options selected by the microprocessor based on information found via the internet, other internal or external information, or the like. Options may be chosen for selecting one or more data feature selections, one or more statistical algorithms, one or more statistical analysis algorithms, one or more statistical significance algorithms, iterative steps, one or more validation algorithms, and one or more graphical representations of methods, machines, apparatuses, computer programs or a non-transitory computer-readable storage medium with an executable program stored thereon.

Systems addressed herein may comprise general components of computer systems, such as, for example, network servers, laptop systems, desktop systems, handheld systems, personal digital assistants, computing kiosks, and the like. A computer system may comprise one or more input means such as a keyboard, touch screen, mouse, voice recognition or other means to allow the user to enter data into the system. A system may further comprise one or more outputs, including, but not limited to, a display screen (e.g., CRT or LCD), speaker, FAX machine, printer (e.g., laser, ink jet, impact, black and white or color printer), or other output useful for providing visual, auditory and/or hardcopy output of information (e.g., outcome and/or report).

In a system, input and output components may be connected to a central processing unit which may comprise among other components, a microprocessor for executing program instructions and memory for storing program code and data. In some embodiments, processes may be implemented as a single user system located in a single geographical site. In certain embodiments, processes may be implemented as a multi-user system. In the case of a multi-user implementation, multiple central processing units may be connected by means of a network. The network may be local, encompassing a single department in one portion of a building, an entire building, span multiple buildings, span a region, span an entire country or be worldwide. The network may be private, being owned and controlled by a provider, or it may be implemented as an internet-based service where the user accesses a web page to enter and retrieve information. Accordingly, in certain embodiments, a system includes one or more machines, which may be local or remote with respect to a user. More than one machine in one location or multiple locations may be accessed by a user, and data may be mapped and/or processed in series and/or in parallel. Thus, a suitable configuration and control may be utilized for mapping and/or processing data using multiple machines, such as in local network, remote network and/or “cloud” computing platforms.

A system can include a communications interface in some embodiments. A communications interface allows for transfer of software and data between a computer system and one or more external devices. Non-limiting examples of communications interfaces include a modem, a network interface (such as an Ethernet card), a communications port, a PCMCIA slot and card, and the like. Software and data transferred via a communications interface generally are in the form of signals, which can be electronic, electromagnetic, optical and/or other signals capable of being received by a communications interface. Signals often are provided to a communications interface via a channel. A channel often carries signals and can be implemented using wire or cable, fiber optics, a phone line, a cellular phone link, an RF link and/or other communications channels. Thus, in an example, a communications interface may be used to receive signal information that can be detected by a signal detection module.

Data may be input by a suitable device and/or method, including, but not limited to, manual input devices or direct data entry devices (DDEs). Non-limiting examples of manual devices include keyboards, concept keyboards, touch sensitive screens, light pens, mouse, tracker balls, joysticks, graphic tablets, scanners, digital cameras, video digitizers and voice recognition devices. Non-limiting examples of DDEs include bar code readers, magnetic strip codes, smart cards, magnetic ink character recognition, optical character recognition, optical mark recognition, and turnaround documents.

A system may include software useful for performing a process or part of a process described herein, and software can include one or more modules for performing such processes. The term “software” refers to computer readable program instructions that, when executed by a computer, perform computer operations. Instructions executable by the one or more microprocessors sometimes are provided as executable code, that when executed, can cause one or more microprocessors to implement a method described herein. A module described herein can exist as software, and instructions (e.g., processes, routines, subroutines) embodied in the software can be implemented or performed by a microprocessor. For example, a module (e.g., a software module) can be a part of a program that performs a particular process or task. The term “module” refers to a self-contained functional unit that can be used in a larger machine or software system. A module can comprise a set of instructions for carrying out a function of the module. A module can transform data and/or information. Data and/or information can be in a suitable form. For example, data and/or information can be digital or analogue. In certain embodiments, data and/or information sometimes can be packets, bytes, characters, or bits. In some embodiments, data and/or information can be any gathered, assembled or usable data or information. Non-limiting examples of data and/or information include a suitable media, pictures, video, sound (e.g., frequencies, audible or non-audible), numbers, constants, values, objects, time, functions, instructions, maps, references, ranges, thresholds, signals, displays, representations, or transformations thereof. A module can accept or receive data and/or information, transform the data and/or information into a second form, and provide or transfer the second form to a machine, peripheral, component or another module. A microprocessor can, in certain embodiments, carry out the instructions in a module. In some embodiments, one or more microprocessors are required to carry out instructions in a module or group of modules. A module can provide data and/or information to another module, machine or source and can receive data and/or information from another module, machine or source.

A computer program product sometimes is embodied on a tangible computer-readable medium, and sometimes is tangibly embodied on a non-transitory computer-readable medium. A module sometimes is stored on a computer readable medium (e.g., disk, drive) or in memory (e.g., random access memory). A module and microprocessor capable of implementing instructions from a module can be located in a machine or in a different machine. A module and/or microprocessor capable of implementing an instruction for a module can be located in the same location as a user (e.g., local network) or in a different location from a user (e.g., remote network, cloud system). In embodiments in which a method is carried out in conjunction with two or more modules, the modules can be located in the same machine, one or more modules can be located in different machine in the same physical location, and one or more modules may be located in different machines in different physical locations.

A machine, in some embodiments, comprises at least one microprocessor for carrying out the instructions in a module. Circadian rhythm factor or circadian rhythm factor metabolite measurement data sometimes are accessed by a microprocessor that executes instructions configured to carry out a method described herein. Circadian rhythm factor or circadian rhythm factor metabolite measurement data that are accessed by a microprocessor can be within memory of a system, and the circadian rhythm factor or circadian rhythm factor metabolite measurement data can be accessed and placed into the memory of the system after they are obtained. In some embodiments, a machine includes a microprocessor (e.g., one or more microprocessors) which microprocessor can perform and/or implement one or more instructions (e.g., processes, routines and/or subroutines) from a module. In some embodiments, a machine includes multiple microprocessors, such as microprocessors coordinated and working in parallel. In some embodiments, a machine operates with one or more external microprocessors (e.g., an internal or external network, server, storage device and/or storage network (e.g., a cloud)). In some embodiments, a machine comprises a module (e.g., one or more modules). A machine comprising a module often is capable of receiving and transferring one or more of data and/or information to and from other modules.

In certain embodiments, a machine comprises peripherals and/or components. In certain embodiments, a machine can comprise one or more peripherals or components that can transfer data and/or information to and from other modules, peripherals and/or components. In certain embodiments, a machine interacts with a peripheral and/or component that provides data and/or information. In certain embodiments, peripherals and components assist a machine in carrying out a function or interact directly with a module. Non-limiting examples of peripherals and/or components include a suitable computer peripheral, I/O or storage method or device including but not limited to scanners, printers, displays (e.g., monitors, LED, LCT or CRTs), cameras, microphones, pads (e.g., ipads, tablets), touch screens, smart phones, mobile phones, USB I/O devices, USB mass storage devices, keyboards, a computer mouse, digital pens, modems, hard drives, jump drives, flash drives, a microprocessor, a server, CDs, DVDs, graphic cards, specialized I/O devices (e.g., photo cells, photo multiplier tubes, optical readers, sensors, etc.), one or more flow cells, fluid handling components, network interface controllers, ROM, RAM, wireless transfer methods and devices (Bluetooth, WiFi, and the like,), the world wide web (www), the internet, a computer and/or another module.

Software often is provided on a program product containing program instructions recorded on a computer readable medium, including, but not limited to, magnetic media including floppy disks, hard disks, and magnetic tape; and optical media including CD-ROM discs, DVD discs, magneto-optical discs, flash memory devices (e.g., flash drives), RAM, floppy discs, the like, and other such media on which the program instructions can be recorded. In online implementation, a server and web site maintained by an organization can be configured to provide software downloads to remote users, or remote users may access a remote system maintained by an organization to remotely access software. Software may obtain or receive input information. Software may include a module that specifically obtains or receives data (e.g., a data receiving module that receives circadian rhythm factor or circadian rhythm factor metabolite measurement data) and may include a module that specifically processes the data (e.g., a processing module that processes received data (e.g., generates Z-scores, generates distributions, provides an outcome and/or report)). The terms “obtaining” and “receiving” input information refers to receiving data by computer communication means from a local, or remote site, human data entry, or any other method of receiving data. The input information may be generated in the same location at which it is received, or it may be generated in a different location and transmitted to the receiving location. In some embodiments, input information is modified before it is processed (e.g., placed into a format amenable to processing (e.g., tabulated)).

Software can include one or more algorithms in certain embodiments. An algorithm may be used for processing data and/or providing an outcome or report according to a finite sequence of instructions. An algorithm often is a list of defined instructions for completing a task. Starting from an initial state, the instructions may describe a computation that proceeds through a defined series of successive states, eventually terminating in a final ending state. The transition from one state to the next is not necessarily deterministic (e.g., some algorithms incorporate randomness). By way of example, and without limitation, an algorithm can be a search algorithm, sorting algorithm, merge algorithm, numerical algorithm, graph algorithm, string algorithm, modeling algorithm, computational genometric algorithm, combinatorial algorithm, machine learning algorithm, cryptography algorithm, data compression algorithm, parsing algorithm and the like. An algorithm can include one algorithm or two or more algorithms working in combination. An algorithm can be of any suitable complexity class and/or parameterized complexity. An algorithm can be used for calculation and/or data processing, and in some embodiments, can be used in a deterministic or probabilistic/predictive approach. An algorithm can be implemented in a computing environment by use of a suitable programming language, non-limiting examples of which are C, C++, Java, Perl, Python, Fortran, and the like. In some embodiments, an algorithm can be configured or modified to include margin of errors, statistical analysis, statistical significance, and/or comparison to other information or data sets (e.g., applicable when using a neural net or clustering algorithm). In some embodiments, an algorithm comprises an algorithm described herein. In some embodiments, an algorithm comprises a Z distribution fit (Raiewski Fit) algorithm described herein.

In certain embodiments, several algorithms may be implemented for use in software. These algorithms can be trained with raw data in some embodiments. For each new raw data sample, the trained algorithms may produce a representative processed data set or outcome. A processed data set sometimes is of reduced complexity compared to the parent data set that was processed. Based on a processed set, the performance of a trained algorithm may be assessed based on sensitivity and specificity, in some embodiments. An algorithm with the highest sensitivity and/or specificity may be identified and utilized, in certain embodiments.

In certain embodiments, simulated (or simulation) data can aid data processing, for example, by training an algorithm or testing an algorithm. Simulated data may be based on what might be expected from a real population or may be skewed to test an algorithm. Simulated data also is referred to herein as “virtual” data. Simulations can be performed by a computer program in certain embodiments. One possible step in using a simulated data set is to evaluate the confidence of identified results, e.g., how well a random sampling matches or best represents the original data. One approach is to calculate a probability value (p-value), which estimates the probability of a random sample having better score than the selected samples. In some embodiments, an empirical model may be assessed, in which it is assumed that at least one sample matches a reference sample (with or without resolved variations). In some embodiments, another distribution, such as a Poisson distribution for example, can be used to define the probability distribution.

A system may include one or more microprocessors in certain embodiments. A microprocessor can be connected to a communication bus. A computer system may include a main memory, often random-access memory (RAM), and can also include a secondary memory. Memory in some embodiments comprises a non-transitory computer-readable storage medium. Secondary memory can include, for example, a hard disk drive and/or a removable storage drive, representing a floppy disk drive, a magnetic tape drive, an optical disk drive, memory card and the like. A removable storage drive often reads from and/or writes to a removable storage unit. Non-limiting examples of removable storage units include a floppy disk, magnetic tape, optical disk, and the like, which can be read by and written to by, for example, a removable storage drive. A removable storage unit can include a computer-usable storage medium having stored therein computer software and/or data.

A microprocessor may implement software in a system. In some embodiments, a microprocessor may be programmed to automatically perform a task described herein that a user could perform. Accordingly, a microprocessor, or algorithm conducted by such a microprocessor, can require little to no supervision or input from a user (e.g., software may be programmed to implement a function automatically). In some embodiments, the complexity of a process is so large that a single person or group of persons could not perform the process in a timeframe short enough for determining one or more characteristics of a sample or subject.

In some embodiments, secondary memory may include other similar means for allowing computer programs or other instructions to be loaded into a computer system. For example, a system can include a removable storage unit and an interface device. Non-limiting examples of such systems include a program cartridge and cartridge interface (such as that found in video game devices), a removable memory chip (such as an EPROM, or PROM) and associated socket, and other removable storage units and interfaces that allow software and data to be transferred from the removable storage unit to a computer system.

Systems, methods, and data structures described herein are operational with numerous other general purpose or special purpose computing system environments or configurations. Examples of known computing systems, environments, and/or configurations that may be suitable include, but are not limited to, personal computers, server computers, thin clients, thick clients, hand-held or laptop devices, multiprocessor systems, microprocessor-based systems, set top boxes, programmable consumer electronics, network PCs, minicomputers, mainframe computers, distributed computing environments that include any of the above systems or devices, and the like. Any type of computer-readable media that can store data that is accessible by a computer, such as magnetic cassettes, flash memory cards, digital video disks, Bernoulli cartridges, random access memories (RAMs), read only memories (ROMs), and the like, may be used in the operating environment.

Provided herein, in certain embodiments, is a system comprising one or more microprocessors and memory, which memory comprises instructions executable by the one or more microprocessors and which memory comprises circadian rhythm factor metabolite measurements from samples from a subject, where the measurements are taken at a plurality of collection times, and which instructions executable by the one or more microprocessors are configured to a) generate a Z score for each collection time according to the corresponding circadian rhythm factor metabolite measurement; b) generate a normal distribution according to the Z scores generated in (a) and the plurality of collection times; and c) generate values for one or more phase markers according to the normal distribution in (b).

Also provided herein, in certain embodiments, is a machine comprising one or more microprocessors and memory, which memory comprises instructions executable by the one or more microprocessors and which memory comprises circadian rhythm factor metabolite measurements from samples from a subject, where the measurements are taken at a plurality of collection times, and which instructions executable by the one or more microprocessors are configured to a) generate a Z score for each collection time according to the corresponding circadian rhythm factor metabolite measurement; b) generate a normal distribution according to the Z scores generated in (a) and the plurality of collection times; and c) generate values for one or more phase markers according to the normal distribution in (b).

Also provided herein, in certain embodiments, is a non-transitory computer-readable storage medium with an executable program stored thereon, where the program instructs a microprocessor to perform the following: a) access circadian rhythm factor metabolite measurements from samples from a subject, where the measurements are taken at a plurality of collection times; b) generate a Z score for each collection time according to the corresponding circadian rhythm factor metabolite measurement; c) generate a normal distribution according to the Z scores generated in (b) and the plurality of collection times in (a); and d) generate values for one or more phase markers according to the normal distribution in (c).

### CERTAIN IMPLEMENTATIONS

Following are non-limiting examples of certain implementations of the technology.

A1. A method comprising:
a) obtaining circadian rhythm factor metabolite measurements from a sample from a subject, wherein the measurements are taken at a plurality of collection times;
b) generating a Z-score for each collection time according to the corresponding circadian rhythm factor metabolite measurement;
c) generating a normal distribution according to the Z-scores generated in (b) and the plurality of collection times in (a); and
d) generating values for one or more phase markers according to the normal distribution in (c).

A2. The method of embodiment A1, wherein the circadian rhythm factor metabolite is a melatonin metabolite.

A3. The method of embodiment A2, wherein the melatonin metabolite is 6-sulfatoxymelatonin (aMT6s).

A4. The method of any one of embodiments A1-A3, wherein the plurality of collection times comprises four or more collection times.

A5. The method of any one of embodiments A1-A4, wherein the collection times are at equal intervals.

A6. The method of any one of embodiments A1-A4, wherein the collection times are at nonequal intervals.

A7. Reserved.

A8. The method of any one of embodiments A1-A7, wherein the measurements are taken at a plurality of collection times during a 24-hour collection period.

A9. The method of any one of embodiments A1-A7, wherein the measurements are taken at a plurality of collection times during a collection period that is less than 24 hours.

A10. The method of any one of embodiments A1-A9, wherein the sample comprises urine.

A11. The method of any one of embodiments A1-A10, wherein the subject is a human.

A12. The method of any one of embodiments A1-A11, further comprising generating a cumulative circadian rhythm factor metabolite measurement for each collection time.

A13. The method of embodiment A12, further comprising generating a total circadian rhythm factor metabolite measurement for a collection period.

A14. The method of embodiment A13, further comprising generating a cumulative proportion of circadian rhythm factor metabolite for each collection time.

A15. The method of embodiment A14, wherein the Z-scores are generated according to the cumulative proportion of circadian rhythm factor metabolite for each collection time.

A16. The method of any one of embodiments A1-A15, wherein the normal distribution is a modified normal distribution.

A17. The method of embodiment A16, wherein the modified normal distribution is produced by a modified normal density function, wherein the modified normal density function includes one or more measured parameters, wherein the one or more measured parameters comprise a total circadian rhythm factor metabolite measurement for a collection period.

A18. The method of any one of embodiments A1-A17, wherein the one or more phase markers comprise one or more of onset, offset, duration, peak value, and peak time.

A19. The method of any one of embodiments A1-A18, further comprising generating a report for the phase marker values generated in (d).

A20. The method of any one of embodiments A1-A19, wherein any or all of (b), (c), and (d) are performed by a microprocessor.

B1. A system comprising one or more microprocessors and memory, which memory comprises instructions executable by the one or more microprocessors and which memory comprises circadian rhythm factor metabolite measurements from samples from a subject, wherein the measurements are taken at a plurality of collection times, and wherein the instructions executable by the one or more microprocessors are configured to:
a) generate a Z-score for each collection time according to the corresponding circadian rhythm factor metabolite measurement;
b) generate a normal distribution according to the Z-scores generated in (a) and the plurality of collection times; and
c) generate values for one or more phase markers according to the normal distribution in (b).

B2. The system of embodiment B1, further comprising one or more features of embodiments A2-A20 and/or further configured to perform one or more methods of embodiments A2-A20.

C1. A machine comprising one or more microprocessors and memory, which memory comprises instructions executable by the one or more microprocessors and which memory comprises circadian rhythm factor metabolite measurements from samples from a subject, wherein the measurements are taken at a plurality of collection times, and wherein the instructions executable by the one or more microprocessors are configured to:
a) generate a Z-score for each collection time according to the corresponding circadian rhythm factor metabolite measurement;
b) generate a normal distribution according to the Z-scores generated in (a) and the plurality of collection times; and
c) generate values for one or more phase markers according to the normal distribution in (b).

C2. The machine of embodiment C1 further comprising one or more features of embodiments A2-A20 and/or further configured to perform one or more methods of embodiments A2-A20.

D1. A non-transitory computer-readable storage medium with an executable program stored thereon, where the program instructs a microprocessor to perform the following:
a) access circadian rhythm factor metabolite measurements from samples from a subject, wherein the measurements are taken at a plurality of collection times;
b) generate a Z-score for each collection time according to the corresponding circadian rhythm factor metabolite measurement;
c) generate a normal distribution according to the Z-scores generated in (b) and the plurality of collection times in (a); and
d) generate values for one or more phase markers according to the normal distribution in (c).

D2. The non-transitory computer-readable storage medium of embodiment D1 further comprising one or more features of embodiments A2-A20 and/or further configured to perform one or more methods of embodiments A2-A20.

E1. A method comprising:
a) obtaining is 6-sulfatoxymelatonin (aMT6s) measurements from urine samples from a human subject, wherein the measurements are taken at four or more collection times;
b) generating a cumulative aMT6s measurement for each collection time;
c) generating a total aMT6s measurement for a collection period;
d) generating a cumulative proportion of aMT6s for each collection time;
e) generating a Z-score for each collection time according to the cumulative proportion of aMT6s;
f) generating a modified normal distribution according to the Z-scores generated in (e) and the four or more collection times in (a); and
g) generating values for phase markers according to the normal distribution in (f), wherein the phase markers comprise onset, offset, duration, peak value, and peak time.

E2. The method of embodiment E1 further comprising one or more features of embodiments A2-A20.

F1. A method comprising:
a) obtaining circadian rhythm factor metabolite measurements from a sample from a subject, wherein the measurements are taken at a plurality of collection times;
b) providing a normal distribution constructed from i) Z-scores generated for each collection time according to the corresponding circadian rhythm factor metabolite measurement and ii) the plurality of collection times in (a); and
c) generating values for one or more phase markers according to the normal distribution provided in (b).

F2. A method comprising:
a) obtaining circadian rhythm factor metabolite measurements from a sample from a subject, wherein the measurements are taken at a plurality of collection times;
b) receiving onto memory a normal distribution constructed from i) Z-scores generated for each collection time according to the corresponding circadian rhythm factor metabolite measurement and ii) the plurality of collection times in (a); and
c) generating, using a microprocessor, values for one or more phase markers according to the normal distribution.

F3. The method of embodiment F1 or F2 further comprising one or more features of embodiments A2-A20.

### EXAMPLES

The examples set forth below illustrate certain implementations and do not limit the technology.

### Example 1: Measuring Melatonin Metabolites According to a Z Distribution Fit

This Example serves to a) describe an algorithm underlying a Z distribution fit method, b) describe parent normal distribution, c) present validation of the Z distribution fit, d) demonstrate estimation of circadian markers, and e) demonstrate superiority of a Z distribution fit over an existing cosine fit analysis under various experimental designs.

### Z Distribution Fit (Raiewski Fit) Method

A Z distribution fit (also referred to as the Raiewski Fit) utilizes properties of a normal distribution to model a frequency-based variable expressing a baseline-elevated period-baseline pattern when sampled over time. This type of profile is typically modeled or “fit” to estimate with precision the timing of the rise (“onset”) and decline (“offset”) and characterize the elevated period regarding length (“duration”) as well as estimate the maximum value (“peak value” or “max value”) and when it occurred (“peak time” or “acrophase”). These parameters may be referred to as “phase markers” in circadian biology. Some examples include nightly wheel running counts in rodents, daily rhythm of sunlight reaching earth (counted in photons), timing of population level gene expression in the mammalian pacemaker through observation of luciferase reporting, and nanograms (ng) of urinary excretion of the melatonin metabolite 6-sulfatoxymelatonin (“aMT6s”) every circadian period. Each of these examples allow for the exact determination of the “total” count of occurrences over the elevated period because each measured variable is considered a frequency within each collection interval. In contrast, collecting blood samples around the clock to measure amounts of melatonin would not result in the ability to estimate the total ng of melatonin secreted, only the timing of relative increases or decreases. However, every circulating melatonin molecule is eventually broken down into aMT6s and excreted in the urine, and when collected, provides an opportunity to obtain the entire nightly count.

The Z distribution fit (Raiewski Fit) begins with user provided (X,Y) coordinate data where X=collection time and Y=counts recorded since the previous collection time. Counts recorded are either conveyed as the total count over the collected interval or as the mean rate within the interval. For example, counting 500 ng aMT6s over a 2-hour interval between 2:00 am and 4:00 am could be expressed as coordinates of (4.0, 500 ng) or (4.0, 250 ng/h) depending on application. Because data such as aMT6s are integrated over time, the Z distribution fit (Raiewski Fit) relies heavily on the assumption the entire nightly total of aMT6s is excreted and collected prior to returning to basal or undetectable levels until the following evening rise. Table 1 below contains real representative data conveying 24-hour collection of urinary aMT6s and the founding computations involved in the Z distribution fit (Raiewski Fit).

TABLE 1
Z distribution (Raiewski Fit) fit algorithm. Input data in the
form of [X, Y] coordinates represent [(column A) decimal
time, (column B) aMT6s ng/h]. Total count of aMT6s ng excreted
per interval are computed (column C) and cumulatively totaled
(column D). Cumulative totals are converted to proportion of total
(column E). Z-score equivalents of cumulative proportion are determined
(column F). Start (i-1) and end (i) time of the collection interval
containing the midpoint (e.g., cumulative proportion crosses 0.5,
where Z = 0) and corresponding Z-scores are used to estimate
σ = (Timei − Timei-1)/(Zi − Zi-1) and μ = Timei − Ziσ.

| | | (C) | (D) | (E) | |
|---|---|---|---|---|---|
| (A) | (B) | aMT6s | aMT6s | Cumultive | (F) |
| Decimal | aMT6s | (ng) per | (ng) | aMT6s | Z-Score |
| Time | (ng/h) | interval | Total | Proportion | Equivalent |
| 20.33 | 1.00 | — | 0.00 | 0 | −∞ |
| 21.83 | 1.00 | 1.50 | 1.50 | 0.000193 | −3.55 |
| 23.32 | 1.00 | 1.49 | 2.99 | 0.000384 | −3.36 |
| 24.79 | 340.05 | 501.58 | 504.57 | 0.064846 | −1.52 |
| 26.32ⁱ⁻¹ | 824.06 | 1256.69 | 1761.26 | 0.226355ⁱ⁻¹ | −0.75ⁱ⁻¹ |
| 28.07ⁱ | 1409.14 | 2465.99 | 4227.25 | 0.543281ⁱ | 0.11ⁱ |
| 29.56 | 1376.06 | 2052.62 | 6279.87 | 0.807082 | 0.87 |
| 30.83 | 317.02 | 401.56 | 6681.43 | 0.858689 | 1.07 |
| 32.31 | 598.17 | 887.29 | 7568.72 | 0.972723 | 1.92 |
| 33.82 | 136.68 | 206.16 | 7774.88 | 0.999218 | 3.16 |
| 35.43 | 1.00 | 1.62 | 7776.50 | 0.999426 | 3.25 |
| 36.96 | 1.00 | 1.53 | 7778.02 | 0.999622 | 3.37 |
| 38.35 | 1.00 | 1.39 | 7779.41 | 0.999801 | 3.54 |
| 39.90 | 1.00 | 1.55 | 7780.97 | 1 | ∞ |

First, any collection interval with 0 secreted aMT6s ng/h is converted to a value of 1 ng/h to avoid computational errors, including dividing by 0, dividing by positive or negative infinity, and redundancy (i.e., ensuring the cumulative total increases each consecutive collection time). Because the primary task is to determine the total nightly ng of aMT6s, this critical alteration only impacts the overall total estimate by less than 0.2%. Table 1, columns A-B, shows this data starts with time×ng/h coordinates, thus aMT6s ng totals are computed at each interval (Table 1, column C) and summed (Table 1, column D) to reveal the nightly ng total of aMT6s excretion (7780.96 aMT6s ng). Once the total aMT6s ng is computed, a cumulative total is calculated for each collection time, and converted to a cumulative proportion (Table 1, column E). Z-scores corresponding to the cumulative proportion are determined at each timepoint (Table 1, column F), taking advantage of an invariant relationship among Z-scores and their fixed location along a normal distribution (e.g., Z Table) defined with population parameters mu (μ) and sigma (σ). A characteristic of the Z distribution fit (Raiewski Fit) is the novel estimation of μ and σ using these Z-scores and their respective collection times (FIG. 1). σ is computed first from two timepoints and the accompanying Z-scores in Table 1 (Rows 5 and 6), where i-1 and i represent the first and second collection time respectively. The rationale of FIG. 1, Equation A, is illustrated with a collection point (i-1) at 0200 when Z=−2.5 and collection point (i) at 0400 when Z=0.5. Here, over the course of 2 hours (0400-0200), 3 standard deviations elapse (0.5-−2.5=3) and would compute σ=0.667. It is important to note any two collection times and their Z-scores (as long as Z is not equal to ±∞) can be used to estimate σ in this way. Because the estimation of u depends on this estimation of σ, and μ is always located at a cumulative proportion of exactly 0.500 (where Z=0.000), i-1 is always selected as the last timepoint with a cumulative proportion less than 0.500 (e.g., the last negative Z-score) and i is the following timepoint which bears a cumulative proportion greater than 0.500 (e.g., the first positive Z-score), ensuring μ is always contained within i-1 and i to avoid extrapolation. For the provided dataset in Table 1, collection time 26.32 is accompanied with a cumulative proportion=0.226 and Z=−0.75 and collection time 28.07 is accompanied with a cumulative proportion of 0.543 and Z=0.11, defining these coordinates as i-1 and i to be used in the first equation where σ=2.0358 (See FIG. 1, Computation A). Next, u is estimated using the standard formula for computing a Z-score, modified to solve for μ, as depicted in FIG. 1, Equation C and Computation D. Traditionally, Zi=(μ-Xi)/σ, however, in this application, Z, X, and σ are known at collection time i, allowing u to be solved for algebraically (μ=27.8454).

### Parent Normal Distribution

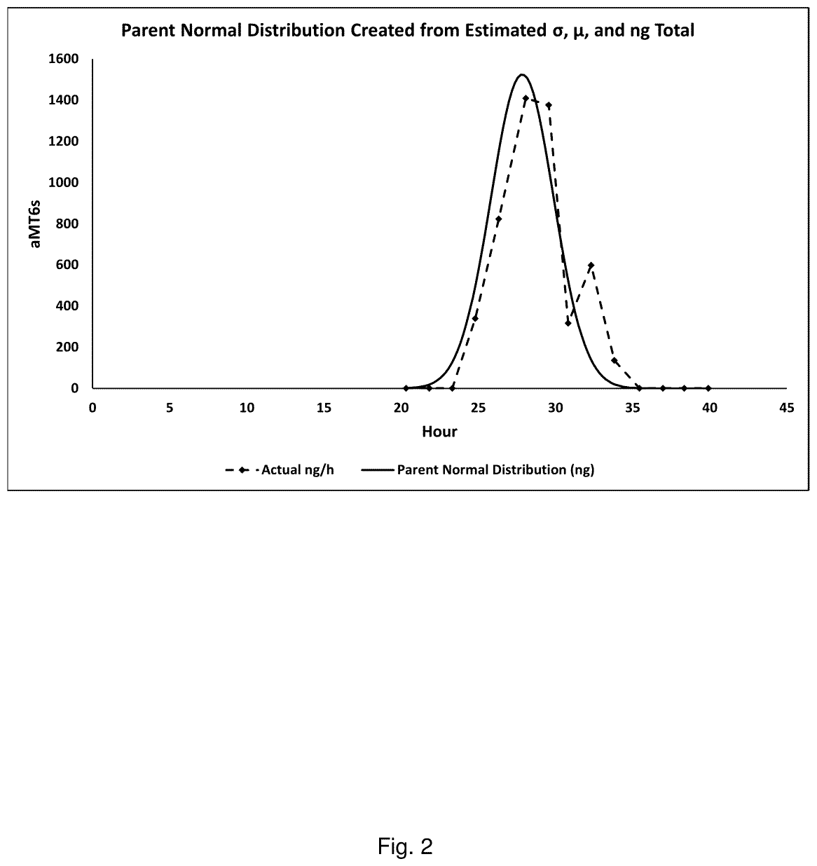

With μ (27.8454) and σ (2.0358) now uniquely estimated, in addition to the nightly total aMT6s ng (7780.97) previously calculated, a modified normal distribution referred to as the “parent normal distribution” is constructed. FIG. 1, Equation C, depicts Carl Gauss's normal distribution function with mean μ and standard deviation σ. FIG. 1, Equation D, introduces the completed function, the parent normal distribution. The parent normal distribution is the central, most important component of the Z distribution fit (Raiewski Fit). The novel manipulation of replacing the 1 in the numerator by the nightly aMT6s total ng (7780.96) sets the area under the curve equal to the nightly aMT6s ng total rather than 1. FIG. 2 plots raw data alongside the constructed parent normal distribution. This parent normal distribution is a histogram used to estimate waveform emerging throughout a complete night of aMT6s excretion, without distortion of collection interval (every 1, 2, 4 hours) or timing (equal intervals or nonequal intervals). Put another way, the parent normal distribution represents the continuous, underlying (i.e., preceding) aMT6s rhythm which urinary collections sample from. The ability for the parent normal distribution to estimate the underlying rhythm rather than directly fit its shape closely to real data, is an unprecedented advancement in circadian biology. The resulting parent normal distribution does not provide a visually satisfying fit, peaking noticeably earlier than raw data. However, this is expected considering the function of the parent normal distribution is indifferent to collection interval timing and frequency, a factor bearing tremendous influence over the resulting plot of raw data (compared to the established method of a cosine fit). Recall the estimation of μ (defined at Z=0, where cumulative proportion=0.500 in a normal distribution) where the peak value (e.g., acrophase) occurs, relies on cumulative proportions at each collection time, not at all where the largest aMT6s ng/h occurs. For the current dataset, the largest raw data value occurs at 28.07 h when the cumulative proportion =0.543, so a peak value corresponding to a cumulative proportion of exactly 0.500 must necessarily occur slightly earlier. Furthermore, the previous datapoint at 26.32 h corresponds to a cumulative proportion of 0.226, allowing one to quickly estimate u necessarily occurs substantially later than 26.32 h and slightly precedes 28.07 h, which it does. Here, μ (27.84 h) is considered analogous to a cosine acrophase, (e.g., peak time) where X=27.84, and, when input to the parent normal distribution function, returns a peak value=1524.78 ng/h, commonly conveyed as peak coordinates (27.84, 1524.78).

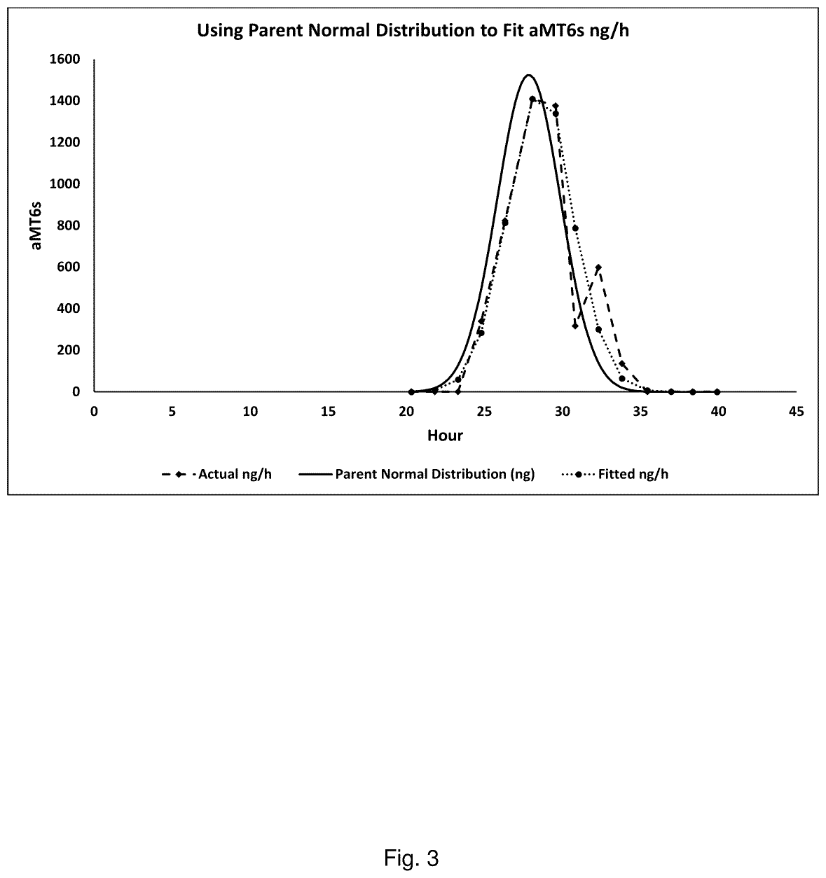

While this resulting parent normal distribution could be evaluated directly on how well it fits raw data, a more accurate fit can be obtained if sampling this parent normal distribution at the same times actual data collection took place. In other words, if sampling the parent normal distribution at the same schedule real data collection actually occurred, and aMT6s counts are converted to mean aMT6s ng/h over each interval (“Fitted ng/h”; see FIG. 3), identical to how actual data is reported and therefore the same units as the input to the Z distribution fit (Raiewski Fit), Pearson's correlation coefficient r and residual standard deviation (RSD, typical difference between actual vs fitted data at each collection time) would be ideal metrics for validating the Z distribution fit (Raiewski Fit). For this particular dataset, the Z distribution fit (Raiewski Fit) returns a correlation of r(11)=0.950 and RSD=164.33 aMT6s ng/h. For comparison, a cosine fit to this same raw data returns r(12)=0.793 and RSD=322.88 aMT6s ng/h, resulting in fits nearly twice as far away (1.96×) from actual data compared to estimates modeled from the Z distribution fit (Raiewski Fit).

### Validation of the Z Distribution Fit (Raiewski Fit)

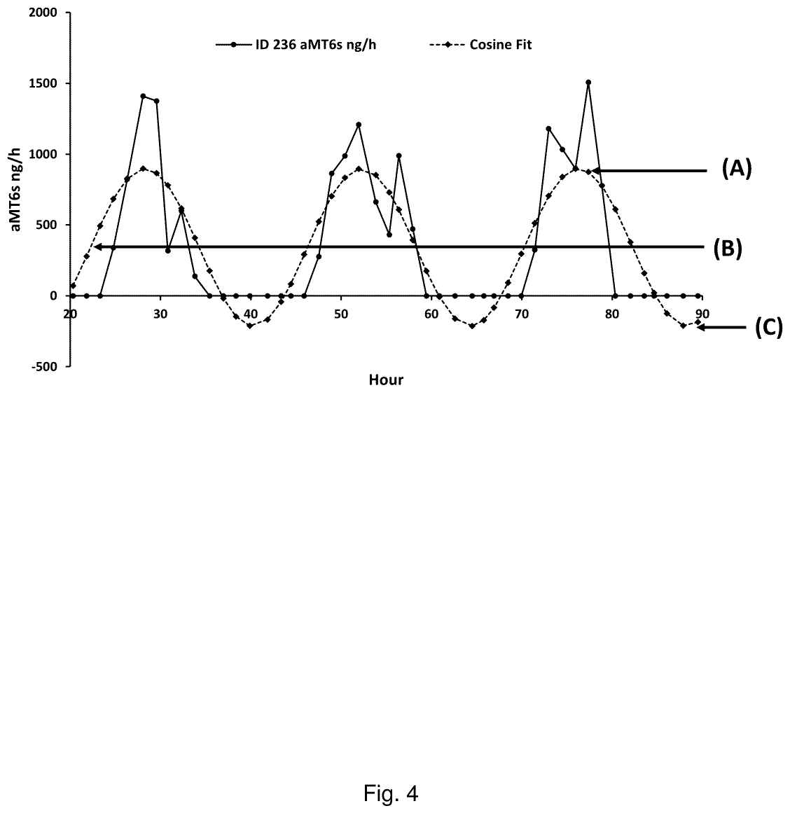

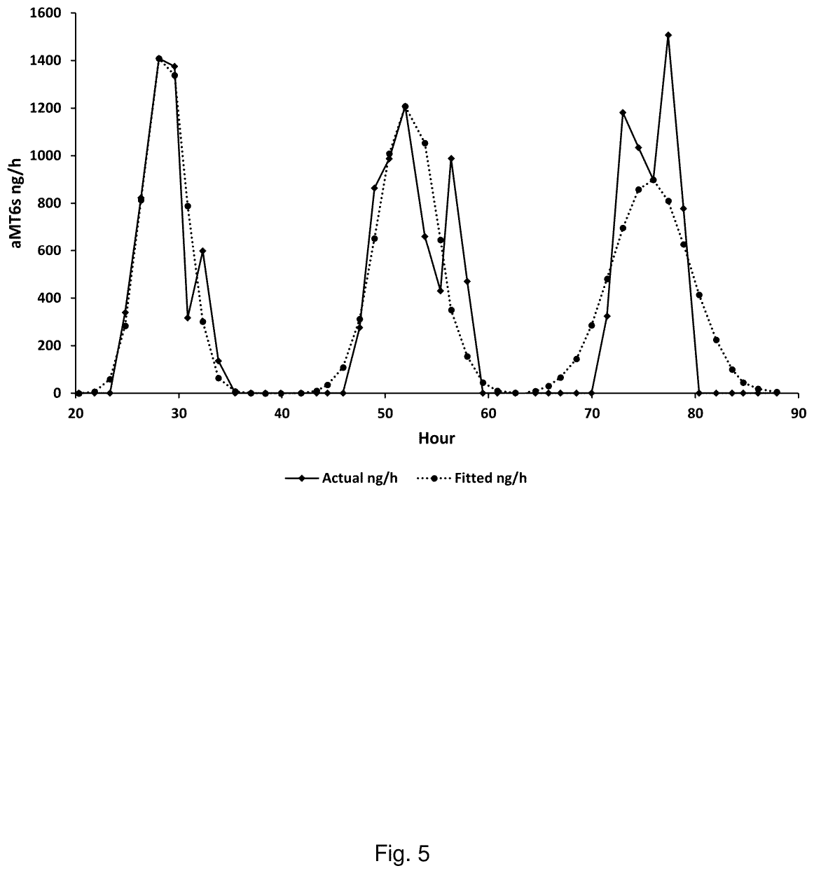

Validation of the Z distribution fit (Raiewski Fit) begins by comparing residual standard deviation (RSD) and Pearson's correlation coefficient (r) to cosine fit results. The RSD quantifies the typical difference between predicted and actual data points for a given fit; Pearson's correlation coefficient, r, quantifies the strength and direction of a relationship between two variables, here assessing the correlation among actual and predicted aMT6s levels. This study uses data from a previously published experiment with consistent 90-minute collection intervals over the span of 3 days in a 90-minute ultra-short sleep-wake protocol. Because accurate total nightly aMT6s totals are necessary for the Z distribution fit (Raiewski Fit), each nightly elevated period of aMT6s excretion must be isolated (e.g., “partitioned”) to individual datasets for separate analysis. To accomplish this, a cosine fit of the complete 3 days is run (see FIG. 4). Each daily partition spans from trough to trough as determined by the period and acrophase from the cosine fit results. For example, a dataset producing an acrophase=25.00 h and period=24.00 h will create a partition centered on the acrophase±half-period, containing all time and data coordinates between 25.00 h±12.00 h (13.00 h-27.00 h). The second daily partition ranges 27.00 h-52.00 h, and 52.00 h-76.00 h the third day, etc. In this way each partition is centered on the fixed 24-hour cosine fit determined acrophase and gives the best chance of capturing 100% of a singular nightly aMT6s excretion total without overlap from the previous or successive nightly elevated aMT6s. For the Z distribution fit (Raiewski Fit), a non-24-hour cosine fit is the standard method for separating multiple days of continuous 90-minute data collection into individual aMT6s partitions. A dataset in the format of baseline-elevated period-baseline is a necessary requirement for the Z distribution fit (Raiewski Fit), satisfied by this method of daily partitioning. A total of N=53 participants recorded for 72 hours at 90-minute collection intervals were included in this validation assessment (see FIG. 5 for a representative single participant using the same actual aMT6s ng/h data as the cosine fit in FIG. 4).

A repeated measures ANOVA was conducted on RSD assessing the factors of Day (1-3) and fit type (cosine, Z distribution) against alpha=0.5. Descriptively, Z distribution fit (Raiewski Fit) yielded lower RSD (mean±standard error: 268.54±46.81) than cosine fit (298.91±53.05), meaning each predicted aMT6s ng/h datapoint is 30.37 ng/h closer to actual data with Z distribution fit (Raiewski Fit) compared to cosine fit, or a 10.2% closer estimate of raw data. This produced a significant main effect of fit type, F (1,50)=9.328, p=0.004. There was no main effect of day (F(2,100)=1.230, p=0.297) or fit type × day interaction (F (2,100)=0.193, p=0.825) an expected finding since day-to-day aMT6s rhythms are assumed to be stable at the individual level. To further illustrate a qualitative improvement of Z distribution fit (Raiewski Fit) over cosine fit, a chi square goodness of fit was conducted to analyze the percent of the time each fit was superior. Given each participant was recorded over three consecutive nights, and that these nights were partitioned into three distinct datasets and analyzed by both cosine fit and Z distribution fit (Raiewski Fit), the superior RSD based fit for each participant was counted. The smaller RSD among cosine fit and Z distribution fit (Raiewski Fit) for each night was obtained for every participant. The fit yielding lowest RSD on the majority of the three partitioned nights (two or more) was determined categorically the best fit for that participant, and results were tallied. For example, if cosine fit yielded a smaller RSD than Z distribution fit (Raiewski Fit) for night 1, but Z distribution fit (Raiewski Fit) produced smaller RSD than cosine fit for nights 2 and 3, the Z distribution fit (Raiewski Fit) is categorically determined the better fit for this participant. Out of 51 participants with all three nights of useable datasets, 33 (64.7%) were overall better fit by the Z distribution, a significant difference at the 0.05 level (X2(1)=4.412, p=0.036).

The same 3×2 repeated measures ANOVA was performed on the Pearson's correlation coefficient r. Z distribution fit (0.865±0.012) correlations were found superior to cosine fit (0.824±0.012), F(1,50)=52.157, p<0.001), without significant day (F(2,100)=1.065, p=0.349) or fit type × day interaction (F(2,100)=0.516, p=0.599). When considering the gain in proportion of variance accounted for (r2), the Z distribution fit (r2=0.748) explains 0.069 greater proportion of the variation of real data than cosine fit (r2=0.679). When categorically assessing which fit was better, 43 of 51 participants (84%) yielded higher correlation coefficients on 2 or more of the three nights with the Z distribution fit compared to cosine fit, a statistically significant outcome (X2(1)=24.0, p<0.001).

One strength of an existing cosine analysis is the ability to analyze multiple days of circadian data with a single cosine fit of each subject. While the above RSD and r results suggest the Z distribution fit is superior to an existing cosine analysis, it is necessary to consider a “traditional” cosine fit would never partition several consecutive days of data into separate 24-hour datasets for individual analysis. In order to compare three partitioned Z distribution fits to a traditional 3-day cosine fit, mean values of RSD and r were computed for each subject from the three separate Z distribution fits (i.e., one from each of three partitioned datasets representing Days 1-3) and compared to RSD and r from the full 3-day (nonpartitioned) cosine fit for each subject (n=53). For RSD, the Z distribution fit (262.3±45.72) was statistically significant compared to cosine fit RSD (335.88±61.50) in a dependent samples t-test (t(52)=3.99, 2 tail p<0.001), revealing each Z distribution fit estimate to be 73.58 aMT6s ng/h closer to each raw datapoint compared to a cosine fit method reflecting the way it is usually performed in real world application (i.e., outside of validation study techniques). In addition, mean RSD of the Z distribution fit was superior to cosine fit RSD in 43 of 53 subjects (81.13%), a complementary significant result (X2(1)=20.6, p<0.001). For r, the Z distribution fit (0.865±0.012) was statistically significant compared to cosine fit (0.762±0.014) in a dependent samples t-test (t(52)=12.14, 2 tail p<0.001). Finally, r values from the Z distribution fit were superior to cosine fit r values in 52 of 53 subjects (98.11%), also a significant finding (X2(1)=49.1, p<0.001). The Z distribution fit reliably fits raw data closer and returns higher correlation coefficients than when cosine fit is run in a traditional (nonpartitioned) way.

Another categorical research advancement the Z distribution fit bears over cosine fit method is the ability to estimate daily period length. A fixed 24 cosine fit rhythm by definition always returns a period of 24 h; However, peak time to peak time in the Z distribution fit provides an estimate of each daily period length, an important advancement in the field, especially given the large quantity of protocols in circadian biology including a “free running” condition where subjects are devoid all environmental timing cues and their natural daily period length is revealed (which is typically >24 h in diurnal and <24 h in nocturnal organisms).

These results establish the Z distribution fit achieves significantly smaller RSD in a majority of subjects compared to existing cosine fit estimates. Significantly higher correlation coefficients are produced with the Z distribution fit in the majority of subjects, demonstrating a higher proportion of variance accounted for with this model over an existing cosine fit. The Z distribution fit demonstrates consistent superiority over cosine fit, in both partitioned and nonpartitioned datasets. These findings together provide reliable validation of the Z distribution fit method. It is also fair to mention these 90-minute intervals of aMT6s data collection over multiple days are the very highest quality resolution a study might hope to carry out and paint a best-case scenario for cosine fit to usefully model data. The attempt to compare the Z distribution fit to the cosine fit under a data collection protocol that would allow the cosine fit model to perform optimally as is done here seemed prudent. However, as collection intervals increase (e.g., fewer samples each 24-hour period) and the number of consecutive days of data collection decrease, cosine fit models become less informative and borderline unreliable. Because the Z distribution fit relies on the total aMT6s counts to estimate the underlying parent normal distribution, the collection interval is incredibly irrelevant, demonstrating continued resiliency under low resolution data collection protocols (further discussed in below).

### Estimation of Circadian Markers

### Cosine Fit and the Z Distribution Equivalents

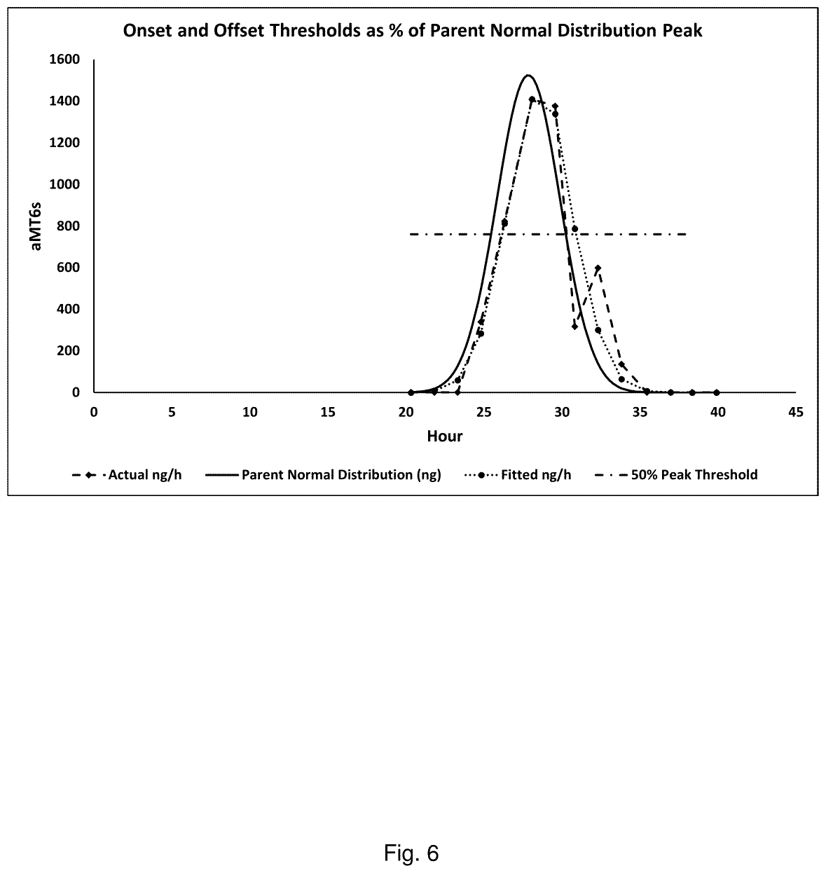

The Z distribution fit (Raiewski Fit) provides several choices for estimating circadian timing of onset, offset, and peak duration (i.e., “elevated duration”). The first method described is most analogous to an existing cosine fit methodology. Briefly, in a cosine fit method, the halfway point between minimum and maximum values of the resulting cosine fit function (i.e., the “mesor”) becomes the vertically based “threshold” value for determining onset and offset. Determining times of crossing points from actual aMT6s ng/h data first crossing above, and last crossing below this level are taken as time of onset and offset, respectively. Peak duration is calculated as the time elapsed between onset and offset. A 50% peak threshold is selected by default for the Z distribution fit because it is most analogous to the 100% mesor, the most commonly adopted threshold in a cosine fit (both are equidistant from minimum and maximum y values generated from their respective models). The dataset in Table 1 returns a parent normal distribution with a peak value of 1524.78 ng, producing a 50% peak threshold of 762.39 ng/h (FIG. 6). To compute the timing of raw data crossing above and below this threshold, straight lines are plotted between adjacent raw data ng/h coordinates, constructed using the point-slope formula. Substitution of the threshold value for Y determines the times of all intersections (crossing points). The first crossing point due to increasing actual aMT6s ng/h defines the time of onset (26.12 h), and the last crossing point due to decreasing actual aMT6s ng/h defines the time of offset (30.29 h). Peak duration equals offset time minus onset time (4.17 h).

Phase Markers Determined from the Parent Normal Distribution

While this peak-based threshold method can be considered the closest equivalent to the determination of circadian markers within a cosine fit, the Z distribution fit (Raiewski Fit) presents an original option—the parent normal distribution—to estimate markers of circadian timing. Recall the parent normal distribution is the foundation of the Z distribution fit (Raiewski Fit) method, defining peak coordinates and generating predicted aMT6s ng/h values with which RSD and r are computed. The same 50% threshold (762.4) can be substituted in for Y in the parent normal distribution function, to locate time of crossing points interpreted as onset (25.45 h) and offset (30.242 h) (FIG. 6). Peak duration is the difference between these crossing point times (4.79 h). Crossing points of the parent normal distribution to a peak value-based threshold provides the advantage of returning exactly two crossing points, whereas actual aMT6s ng/h data are often noisy and cross above and below the threshold repeatedly, an occurrence introducing additional error into this method of phase marker selection. The drawback to using the parent normal distribution for estimating circadian markers is the onset and offset are always equidistant from u and may not faithfully reflect the waveform of subjects demonstrating skewed or asymmetric aMT6s excretion profiles. This same problem is always apparent if using the cosine wave computed in a cosine fit; a cosine wave crosses the mesor exactly twice, and worse, because the period is always 24 h, these crossing points occur exactly every 12 hours. Hence the method for determining phase markers with the cosine fit relies on crossing points of actual ng/h data rather than the cosine function directly.

### Phase Markers Using Fitted Ng/h Estimates

The Z distribution fit (Raiewski Fit) provides a novel and improved method for employing a peak threshold-based determination of onset and offset: use the crossing points of fitted ng/h data rather than actual aMT6s ng/h data. In contrast to actual aMT6s ng/h data, fitted ng/h data will produce exactly one onset time and one offset time, occurring at 26.168 h and 30.905 h in FIG. 6. While this method ensures exactly 2 crossing points, this method does not require they occur at equidistant magnitudes from u as is the case if using crossing points from this threshold directly from the parent normal distribution. Cosine fit model has no equivalent metric.

While the mesor value (or the 50% peak value) is a typical threshold level, the reasoning behind this adoption is out of necessity rather than because it meets theoretical satisfaction. This is because the ideal phase markers would determine onsets at times most closely corresponding to the initial significant elevation of aMT6s from baseline, and offsets with closest temporal correspondence to the final aMT6s decline back to basal levels. However, subject data typically have “noisy” baseline values, and if using a lower threshold in an attempt to increase sensitivity to initial increase and final declines, the likelihood of multiple crossing points increases. The higher the threshold, the less likely multiple crossing points will be returned due to noisy baseline data collection. Determining onset and offset phase markers using crossing points from fitted ng/h data are not affected by noisy baselines while maintaining extremely close estimates of the actual data. As will be discussed in depth below, this allows for a lower threshold to be selected that can provide phase marker estimates more temporally aligned to initial increase from baseline and final return to baseline, a major advancement.

### Superiority of the Z Distribution Fit (Raiewski Fit)

### Nonbiased Estimate of Peak Values

The Z distribution fit (Raiewski Fit) produces a biologically realistic fit compared to existing cosine fitting. First, the Z distribution fit has inherent basal levels prior to and following nightly rise of aMT6s excretion, while cosine fits do not have the capacity to predict prolonged periods of basal/non-detectable values. Second, the Z distribution fit cannot predict a value below zero-a biological impossibility-whereas cosine fits regularly predict aMT6s levels in the negative range. Cosine fit values rely on generating a function minimizing the sum of squared residuals (e.g., least squares) among actual and predicted data. As a result, peak values are most often substantially underestimated. The Z distribution fit merely computes a normal distribution function based on the 50th percentile of the nightly total of aMT6s metabolite, providing a superior approximation of peak aMT6s amplitude. Said another way, cosine fitting is achieved through purposeful matching of high and low data points to estimates, whereas the peak value in the Z distribution fit is achieved by matching the volume of a normal distribution of a given μ and σ to an equivalent aMT6s ng total. Moreover, existing cosine fit places primary importance on determining a cosine function that numerically fits the raw data, whereas visually satisfying curves modeled by the Z distribution fit are merely a biproduct of the underlying parent normal distribution. Looking again at the 3-day study, actual nightly maximum aMT6s ng/h values for each night were compared to estimated peak aMT6s values from cosine and Z distribution fits (comparing estimated maximum values from FIG. 4-5). Only 4 of 159 (2.52%) cosine fits predicted a peak value larger than the actual maximum value, underestimating the true maximum by a median of 186 aMT6s ng/h, on average only reaching 77% of the true maximum value, a substantial concern (X2(1)=143.40, p<0.001). In contrast, 74 of 159 (46.54%) Z distribution fit predicted peak values were greater than the true nightly maximum, underestimating the maximum by a median of 9.84, at a rate not significantly different from chance (X2(1)=0.761, p<0.383). Remarkably, this suggests the Z distribution fit-determined peak aMT6s values are unbiased estimates of the real nightly maximum, a vast advancement. This is especially valuable when considering the threshold value for determining circadian timing of onset, offset, and duration is directly proportionate to this estimated peak amplitude. Additionally, by sampling via discrete collection times which result in a mean rate (ng/h) per interval, the peak amplitude will always be underestimated since the peak will be contained (e.g., averaged in) within an interval reflecting a value that more heavily weights the lesser amplitudes preceding and proceeding it.

### Indifference to Collection Interval

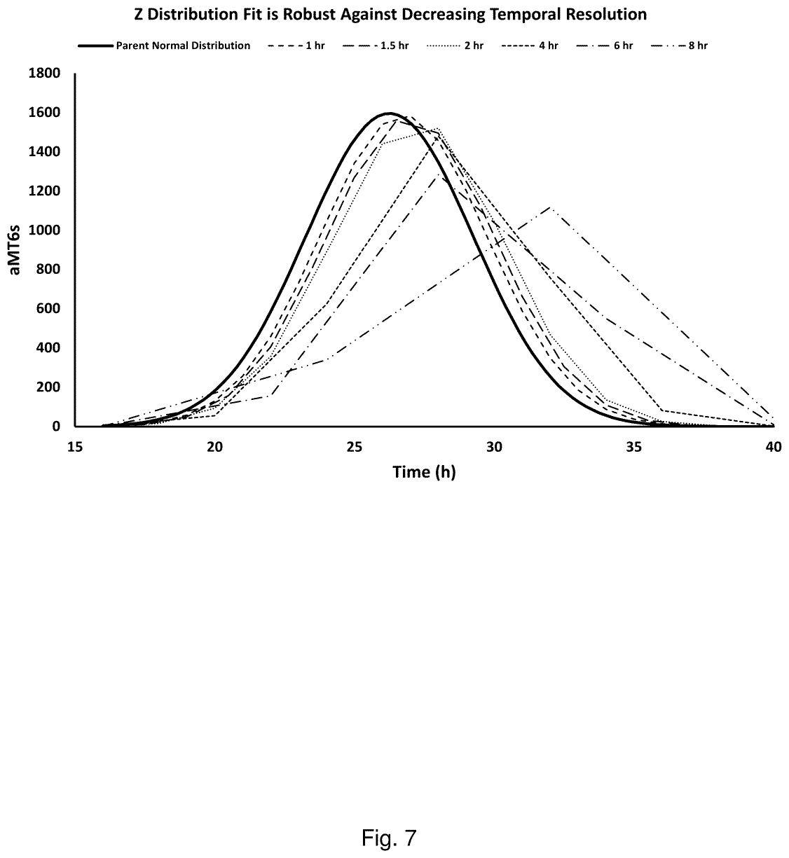

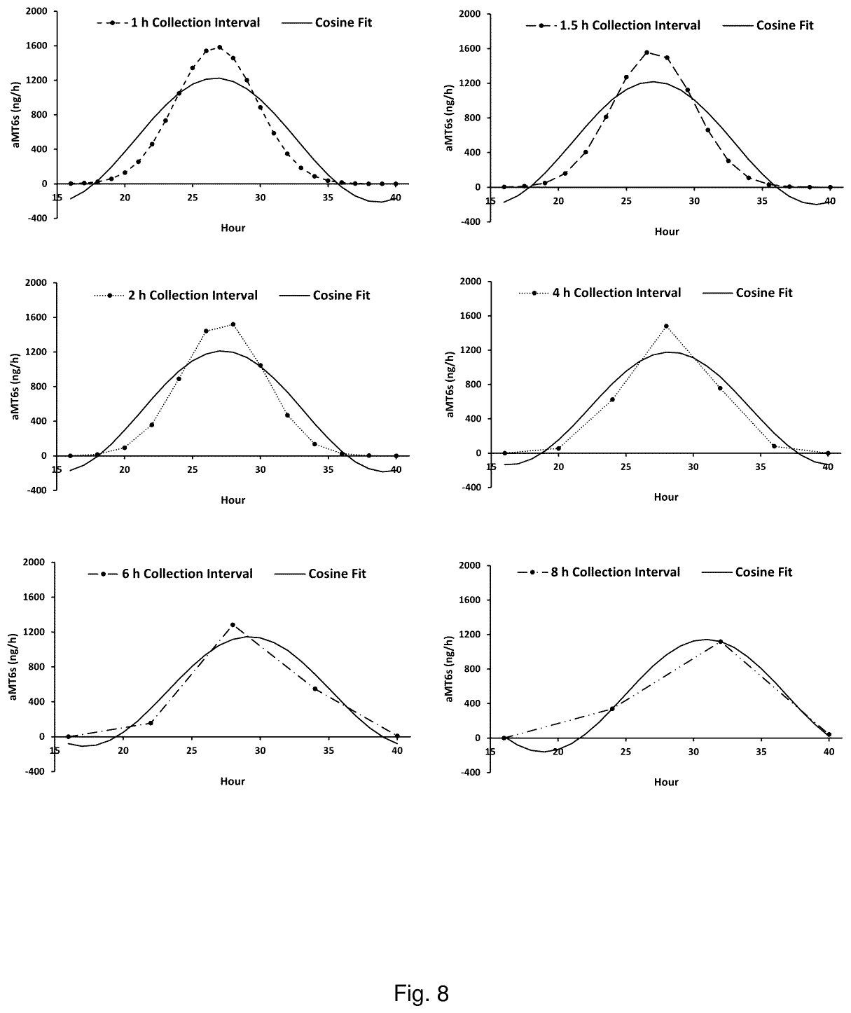

One major novel feature of the Z distribution fit (Raiewski Fit) is how fitted aMT6s ng/h estimates are derived from the parent normal distribution. This method is indifferent to collection interval timing and spacing because proportions under the curve of total nightly aMT6s ng are integrated over time. This concept can be illustrated when considering a night of aMT6s excretion with a normally distributed waveform. For example, consider a nightly aMT6s total of 12,000 ng, with μ=26.25 (02:15 am) and σ=3.00 (“example parent normal distribution”). In an attempt to simulate the distortions of resulting measurements in real studies, the example parent normal distribution is sampled over collection intervals of 1, 1.5, 2, 4, 6, and 8 h (FIG. 7). The result is attenuated and delayed peak values as collection interval increases. Problems arise when these profiles are cosine fit (FIG. 8; Table 2).

TABLE 2
Cosine fit results are highly sensitive to collection interval.
Resulting parameters as calculated by traditional cosine
fit to hypothetical data collection from previously described
example parent normal distribution. As collection interval
(column A) increases over a 24 hr study, fewer data points
(column B) are collected. Cosine fit mesor (column C), amplitude
(column D), acrophase (column E) and peak aMT6s ng/h value
(column F) are the parameters computed from a cosine fit.
Every parameter is sensitive to collection interval.

| (A) | | | | | (F) |
|---|---|---|---|---|---|
| Collection | (B) | (C) | (D) | (E) | Peak aMT6s |
| Interval | Data Points | Mesor | Amplitude | Acrophase | (ng/h) |
| 1 hr | 25 | 507.17 | 718.95 | 26.73 | 1226.12 |
| 1.5 hr | 17 | 510.79 | 709.07 | 26.97 | 1219.86 |
| 2 hr | 13 | 514.15 | 698.5 | 27.22 | 1212.65 |
| 4 hr | 7 | 522.23 | 657.21 | 28.27 | 1179.44 |
| 6 hr | 5 | 519.52 | 629.24 | 29.22 | 1148.76 |
| 8 hr | 4 | 493.01 | 562.054 | 30.91 | 1055.06 |

A systematic bias decreases cosine fit estimated mesor, and as a result, the 100% mesor threshold and corresponding phase markers drift unreliably, as they are dependent upon collection interval. Because the nightly aMT6s ng total is not affected by collection interval, the Z distribution fit resiliently returns estimated parameters of the example parent normal distribution with precision and robust indifference to collection schedule (Table 3).

TABLE 3
Z distribution is robust and unaffected by collection interval.
Resulting parameters as calculated by Z distribution fit (Raiewski
Fit) to hypothetical data collection from previously described
example parent normal distribution (μ = 26.25 h, σ =
3.0 h, nightly total aMT6s count = 12,000 ng) at 1, 1.5,
2, 4, 6, and 8 h intervals. As collection interval (column A)
increases over a 24 hr study, fewer data points (column B) are
collected. Parameters of the parent normal distribution returned
for each collection interval (μ (column C), σ (column
D), peak aMT6s ng value (column E), and nightly total aMT6s count
(column F) are not affected by collection interval. All collection
times uniformly return highly accurate estimates of the example
parent normal distribution.

| (A) | | | | (E) | (F) |
|---|---|---|---|---|---|
| Collection | (B) | (C) | (D) | Peak | Total Count |
| Interval | Data Points | μ | σ | aMT6s (ng) | aMT6s (ng) |
| 1 hr | 25 | 26.251 | 2.999 | 1595.72 | 11996.17 |
| 1.5 hr | 17 | 26.251 | 2.999 | 1595.9 | 11996.17 |
| 2 hr | 13 | 26.251 | 2.999 | 1595.65 | 11996.17 |
| 4 hr | 7 | 26.252 | 2.999 | 1595.92 | 11996.17 |
| 6 hr | 5 | 26.252 | 2.997 | 1596.65 | 11996.17 |
| 8 hr | 4 | 26.252 | 2.999 | 1595.68 | 11996.17 |

### Indifference to Limited Data

Another advantage of the Z distribution fit (Raiewski Fit) is indifference to internal or expected period. The Z distribution fit does not require either an assumption of a 24 h period or to solve for a determined period in order to complete the fit or compute circadian markers, whereas cosine fit either assumes data are generated on a 24-hour rhythm or must solve for the period prior to computing circadian markers. With Z distribution fit, internal period (tau) can be computed through a regression line involving either peak to peak, onset to onset, or offset to offset times in the same way as existing cosine fitting with fixed 24 h period. Because the Z distribution fit relies exclusively on proportions of a single nightly aMT6s ng total, a complete, reliable, accurate sample can be obtained in under 24 hours, if urine collection begins prior to early evening rise (2000) and ends after the final aMT6s metabolite is excreted later the following morning (1200). Resiliency of the Z distribution fit is most apparent under collection schedules demanding unequal and/or infrequent collection times. For example, the verify study protocol included urine collection precisely every 90 minutes over a total of 72 hours for a total of 48 collection times. With the Z distribution fit, reliable estimates can be obtained with as little as four unevenly spaced collection times, encouraging participant friendly home-collected data protocols (early evening, right before bed, directly upon waking, and around noon) with the critical assumptions that a) participants fully void at each urination, and b) the 50th percentile of aMT6s excretion occurs between the second and third collection (while sleeping). In other words, any sampling schedule yielding a baseline, elevated, elevated, baseline result can usefully employ the Z distribution fit algorithm. The benefit of obtaining reliable aMT6s data with as little as four collection times, in as little as 18 hours or less—from home, is an unprecedented capability that cannot be overstated. The Z distribution fit can provide the means necessary toward shifting the paradigm of cumbersome, multi-day in-patient study designs toward a more participant centered approach in line with what many IRBs have more recently encouraged.

Additional Metrics Inferred from the Area Under the Curve of a Normal Distribution

The fixed relationship between Z-scores and proportions under a normal distributions creates several novel metrics the parent normal distribution can offer in addition to phase markers derived from a peak value. Because any peak-based threshold value chosen will result in crossing points on the parent normal distribution at locations equidistant from the center (u), these crossing points will always correspond to Z-scores of equal magnitudes. For example, a 25% threshold produces crossing points at Z-scores of ±1.67, a 50% threshold for any dataset will always produce crossing points on the parent normal distribution at Z-scores of ±1.177, and a 75% threshold produces crossing points at Z-scores of ±0.76. This fixed relationship between peak determined thresholds and Z-scores are important for two reasons. First, the area under the curve of the parent normal distribution during peak duration can be readily determined, and a proportion of the nightly total aMT6s excreted during the peak duration can be computed. FIG. 9 illustrates this feature of the Z distribution fit algorithm, again drawing from parameters of the example parent normal distribution. Here, a 50% threshold crosses the parent normal distribution at Z-scores of ±1.177, and 76.1% of the area under the curve of any normal distribution is contained within this interval. Taking 76.1% of the total aMT6s ng produced reveals the proportion of aMT6s ng excreted during the peak duration.

### Sigma as an Inherent Threshold

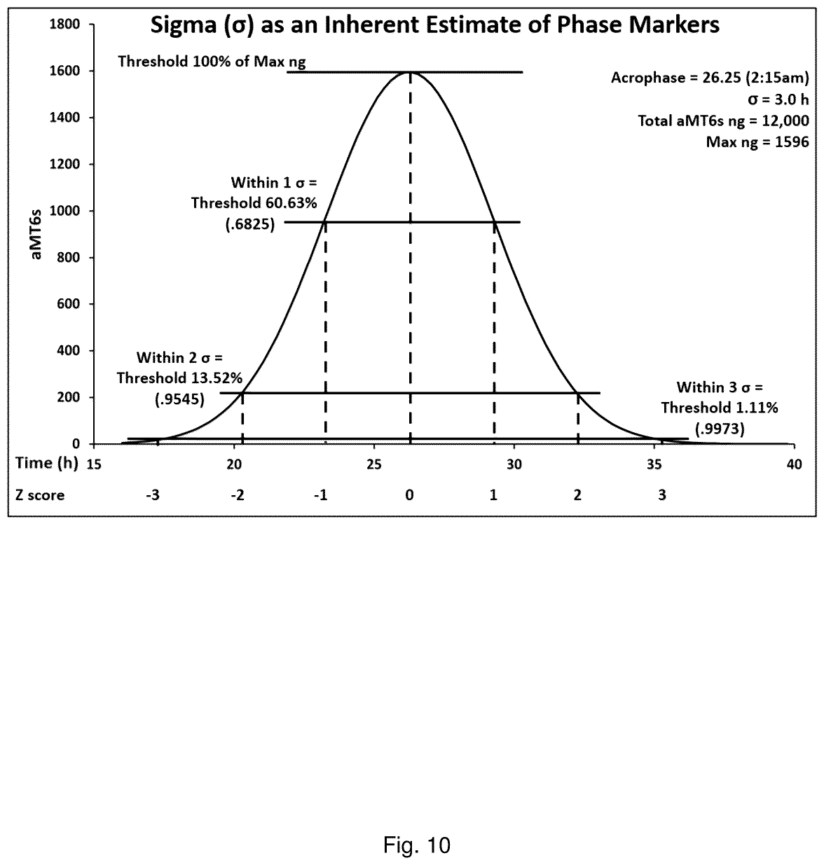

An additional novel advantage the Z distribution fit (Raiewski Fit) provides is an alternative metric for quantifying peak duration which does not rely on arbitrary, or subjective, threshold values. Sigma (σ), the population standard deviation, is an inherent parameter of the normal distribution. This is illustrated using proportions under the curve of the example parent normal distribution (FIG. 10). Sigma is the singular parameter defining the width of a normal distribution, and the first parameter solved for in the Z distribution fit method. Sigma and threshold values operate under the same principles for determining onset and offset, with one important distinction: The selected threshold value is subjective, but the determination of sigma is not. Importantly, σ (or variance, σ²) has inherent standalone value in quantifying elevated aMT6s duration, as σ is the singular determining parameter for the width of a normal distribution and where the width of the normal distribution within the context of the Z distribution fit is time. Importantly, this introduces the use of Z-scores and their fixed proportions under a normal distribution as a superior criterion for determining onset and offset of elevated values. In other words, onset and offset could be defined as the interval of the parent normal distribution contained within 1 standard deviation of μ, containing the middle 68.25% of the total nightly aMT6s ng, equivalent to selecting a peak-based threshold of 60.63%. A threshold of 2 standard deviations from u creates an interval containing the middle 95.45% of the total aMT6s ng, equivalent to selecting a peak-based threshold of 13.52%. A threshold within 3 standard deviations of u would contain the middle 99.73% of total nightly aMT6s ng and would be equivalent to selecting a peak-based threshold of 1.11%. Alternatively, proportions under the curve of a normal distribution commonly representing a values could be the basis of selection: for α=0.05, the middle 95% of total nightly aMT6s ng is contained within ±1.96 standard deviations from μ, equivalent to selecting a peak-based threshold of 14.65%, and for α=0.01, the middle 99% of total nightly aMT6s ng is contained within +2.58 standard deviations from u, equivalent to selecting a peak-based threshold of 3.62%.

The ideal solution to proper selection of the above suggested thresholds in determining the phase markers of onset and offset is to use the timing of fitted ng/h crossing points to a set factor of σ. This would eliminate the most common problem accompanying actual ng/h crossing points: noisy baseline data typically results in multiple crossings above and below the desired threshold value. Using fitted ng/h data ensures exactly one threshold crossing on the way up to the peak and exactly one crossing below the threshold value upon return to baseline. This method of determining onsets and offsets is also advantageous over the parent normal distribution fit because it does not force onset and offset times to remain equidistant to the peak. Taking the threshold as a factor or σ (i.e., 13.52% to reflect aMT6s excretion within 20) integrates the advantages of all three onset/offset criteria methods without taking on any of the disadvantages while providing the ability to assess phase markers at the theoretically ideal lower threshold.

### Superior Alternative to Accounting for Errant Data and Outliers

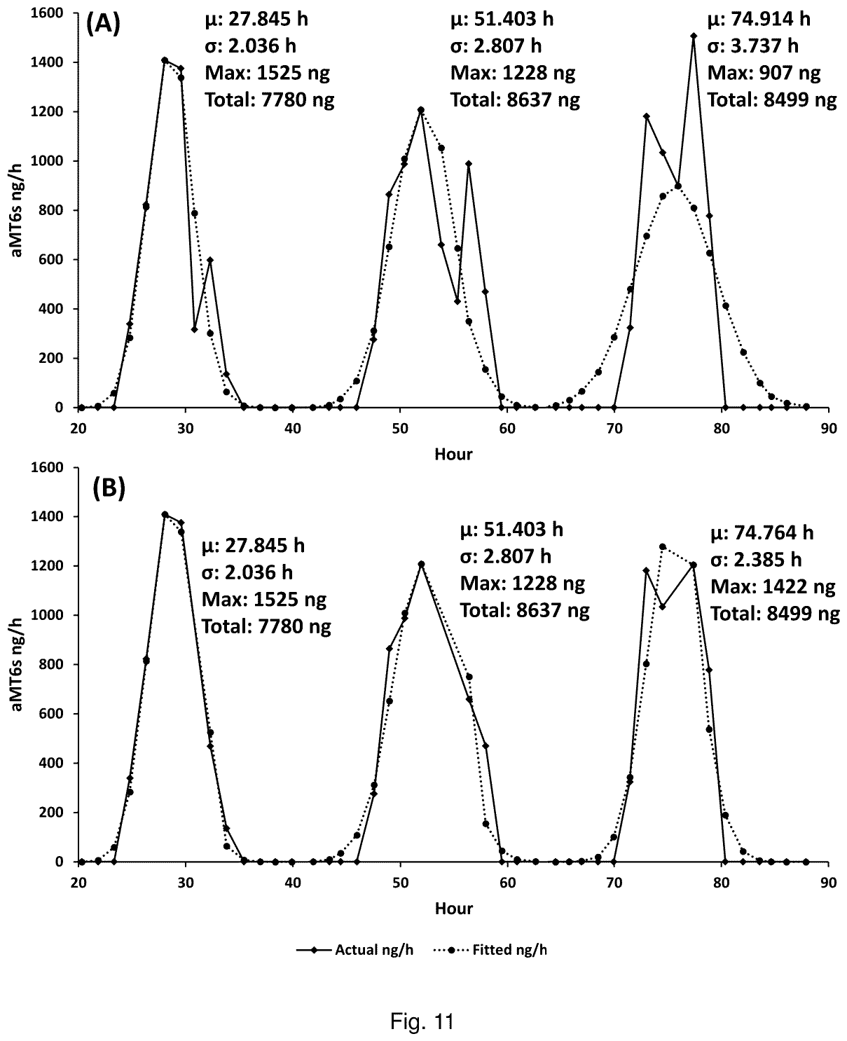

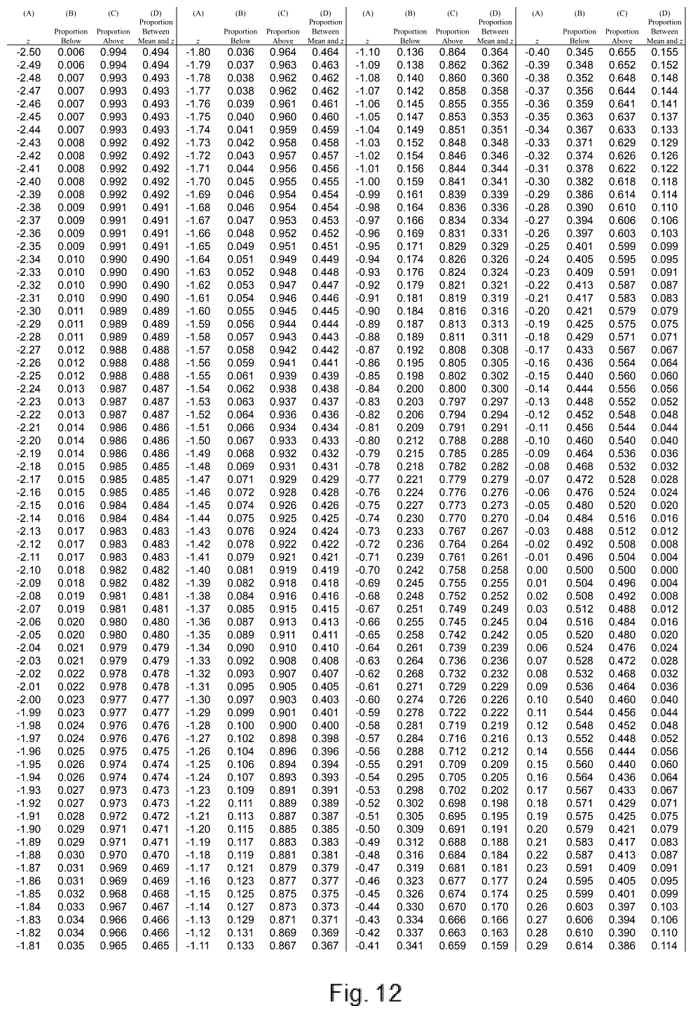

Another novel advantage of the Z distribution fit (Raiewski Fit) method regards the option to combine temporally adjacent collection points as a means of correcting errant values. The primary source of error during collection of urinary metabolite aMT6s is the failure of the patient to void completely. When this happens the resulting dataset displays a very low value, followed by a very high value due to the patient completely voids at the time of the next collection, which is now a combination of newly metabolized aMT6s plus the remainder of the prior collection time. Each of these collection points are erroneous, but can be corrected if combined into a single collection point representing the latter time. For example, a patient voids completely at 1:00 am, then incompletely at 2:30 am, then completely at 4:00 am, the 2:30 am value sticks out as extremely low and the 4:00 am value sticks out as extremely high with respect to the remainder of the collection times sampling this night of aMT6s waveform. To account for this the aMT6s ng from 2:30 am and 4:00 am must be combined into a 3-hour collection interval, resulting in a collection simulating as if the patient had waited three hours to void at 4:00 am rather than twice every 90 minutes. For existing cosine analysis this affects the equal interval spacing carefully constructed in the experimental design, and reduces the number of useable datapoints necessary to properly fit the cosine function—an action known to distort results (see above, indifference to collection interval). The benefit to the Z distribution fit method is that the cumulative proportions prior to 2:30 am and after 4:00 am remain exactly the same. Because the parent normal distribution function relies only on the cumulative proportions of the nightly total at each collection interval, as long as the 50th percentile was not crossed between 1:00 am-2:30 am, the 4:00 am cumulative proportion, and subsequent cumulative proportions, are unaffected. This data cleaning method is illustrated in FIG. 11.

### Note for Data Collection Practice

Data preparation for using the Z distribution fit (Raiewski Fit) method is suggested to list mean ng/h within each collection interval, but would be most precise if conveyed at actual collection times rather than the midpoint between two collection points, as existing cosine analysis requires. A minimum of four collection times, with elevated aMT6s values before and after the 50th percentile of the cumulative total are required for successful Z distribution fit results. One source of Z distribution failure comes from not having enough elevated datapoints to identify u. This occurrence is reduced in the following two ways when partitioning data files. First, for partitioned days 2 and beyond, the last coordinate of the previous partition is included as the first coordinate. This can be achieved because very first plotted aMT6s ng/h already considers the duration of time since the prior collection time to get ng/h. The very first aMT6s ng/h value in a partitioned dataset is replaced with “1” as it is never incorporated into the Z distribution fit but only contributes the initial timepoint to be used to convert the second aMT6s ng/h coordinate into total aMT6s ng produced within the interval. As such, this does not manufacture additional datapoints but rather incorporates information already used for conversion to ng/h. Second, any values=0 aMT6s ng/h values are replaced with 1 aMT6s ng/h. This replacement ensures the only coordinates associated with cumulative proportions of 0 and 1 are the first and last coordinates, respectively. Cumulative proportions of 0 and 1 result in Z-scores of −∞ and ∞, respectively, the only two values that cannot be substituted in to compute μ or σ. These two minor alterations of partitioned datasets increase the likelihood that the last coordinate prior to and first coordinate after cumulative proportion =0.500 are cumulative values greater than 0 and less than 1, a necessary requirement for the Z distribution fit to arrive at a solution.

Taken together, the case for validation by this Example indicates the Z distribution fit is a resilient, elegant, and superior method for fitting circadian data. The elementary foundation of counting the total nightly excretion of aMT6s ng makes it a quantitative advancement in every comparable way to the cosine fit. Compared to conventional cosine fit methods, the Z distribution fit provides superior fits of data, with fewer data points and with unequal collection intervals. The Z distribution fit does not require 24 hours of data collection. Accordingly, home collection protocols may be developed and implemented which massively benefit both participants and researchers due to the reduced and flexible collection schedule. The Z distribution fit creates an estimate of the underlying rhythm producing collected data, the parent normal distribution, a manipulation with no equal. The estimated peak value in a Z distribution fit is demonstrated to be an unbiased estimate of the actual ng/h data. The Z distribution fit returns estimated values that are biologically realistic (i.e., peak values reliably represent actual data, no estimated values below 0) compared to cosine fit. The Z distribution fit develops a superior method for obtaining vital phase markers using fitted ng/h values. The Z distribution fit takes advantage of known proportions and their relationship to Z-scores under a normal distribution, creating an unprecedented approach to quantifying phase markers, through the inherent index of the width of σ, the defining parameter of width in a normal distribution. Merging errant datapoints improves, rather than distorts, resulting estimates. Moreover, the Z distribution fit can be applied to any field or method of data collection obtaining a total temporal count going from baseline to elevated period to baseline in a normally distributed manner.

The technology described herein may be used for various clinical and research purposes. For example, precise measurements of circadian markers may be used to look for differences in circadian timing (or chronophase) among different cohorts or demographics, for example: men vs. women, old vs. young, pre-menopause vs. post-menopause. In certain instances, precise measurements of circadian markers may be used to look at numerous disorders (e.g., depression, mania, insomnia, schizophrenia, etc.) to establish predictable deviations compared to parameters of the larger population. Additionally, precise measurements of circadian markers may be useful for research that looks at how individuals adapt under numerous entrainment protocols, to determine the magnitude of phase shifting (advancing or delaying) as a result of treatment (e.g., measuring the difference in acrophase pre and post treatment to determine the phase shift elicited).

The entirety of each patent, patent application, publication and document referenced herein is incorporated by reference. Citation of patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

The technology has been described with reference to specific implementations. The terms and expressions that have been utilized herein to describe the technology are descriptive and not necessarily limiting. Certain modifications made to the disclosed implementations can be considered within the scope of the technology. Certain aspects of the disclosed implementations suitably may be practiced in the presence or absence of certain elements not specifically disclosed herein.

Each of the terms “comprising,” “consisting essentially of,” and “consisting of” may be replaced with either of the other two terms. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%; e.g., a weight of “about 100 grams” can include a weight between 90 grams and 110 grams). Use of the term “about” at the beginning of a listing of values modifies each of the values (e.g., “about 1, 2 and 3” refers to “about 1, about 2 and about 3”). When a listing of values is described the listing includes all intermediate values and all fractional values thereof (e.g., the listing of values “80%, 85% or 90%” includes the intermediate value 86% and the fractional value 86.4%). When a listing of values is followed by the term “or more,” the term “or more” applies to each of the values listed (e.g., the listing of “80%, 90%, 95%, or more” or “80%, 90%, 95% or more” or “80%, 90%, or 95% or more” refers to “80% or more, 90% or more, or 95% or more”). When a listing of values is described, the listing includes all ranges between any two of the values listed (e.g., the listing of “80%, 90% or 95%” includes ranges of “80% to 90%,” “80% to 95%” and “90% to 95%”).

Certain implementations of the technology are set forth in the claim(s) that follow(s).

What is claimed is:

1. A method comprising:
a) obtaining circadian rhythm factor metabolite measurements from a sample from a subject, wherein the measurements are taken at a plurality of collection times;
b) generating a Z-score for each collection time according to the corresponding circadian rhythm factor metabolite measurement;
c) generating a normal distribution according to the Z-scores generated in (b) and the plurality of collection times in (a); and
d) generating values for one or more phase markers according to the normal distribution in (c).

2. The method of claim 1, wherein the circadian rhythm factor metabolite is a melatonin metabolite.

3. The method of claim 2, further comprising administering melatonin according to the values for the one or more phase markers generated in (d).

4. The method of claim 2, wherein the melatonin metabolite is 6-sulfatoxymelatonin (aMT6s).

5. The method of claim 1, wherein the plurality of collection times comprises four or more collection times.

6. The method of claim 1, wherein the collection times are at equal intervals.

7. The method of claim 1, wherein the collection times are at nonequal intervals.

8. The method of claim 1, wherein the measurements are taken at a plurality of collection times during a 24-hour collection period.

9. The method of claim 1, wherein the measurements are taken at a plurality of collection times during a collection period that is less than 24 hours.

10. The method of claim 1, wherein the sample comprises urine.

11. The method of claim 1, wherein the subject is a human.

12. The method of claim 1, further comprising generating a cumulative circadian rhythm factor metabolite measurement for each collection time.

13. The method of claim 12, further comprising generating a total circadian rhythm factor metabolite measurement for a collection period.

14. The method of claim 13, further comprising generating a cumulative proportion of circadian rhythm factor metabolite for each collection time.

15. The method of claim 14, wherein the Z-scores are generated according to the cumulative proportion of circadian rhythm factor metabolite for each collection time.

16. The method of claim 1, wherein the normal distribution is a modified normal distribution.

17. The method of claim 16, wherein the modified normal distribution is produced by a modified normal density function, wherein the modified normal density function includes one or more measured parameters, wherein the one or more measured parameters comprise a total circadian rhythm factor metabolite measurement for a collection period.

18. The method of claim 1, wherein the one or more phase markers comprise one or more of onset, offset, duration, peak value, and peak time.

19. The method of claim 1, further comprising generating a report for the phase marker values generated in (d).

20. The method of claim 1, wherein any or all of (b), (c), and (d) are performed by a microprocessor.
